(19) United States
(12) Patent Application Publication
(10) Pub. No.: US 2023/0383342 A1
(43) Pub. Date: Nov. 30, 2023

(54) COMPOSITIONS AND METHODS FOR NUCLEIC ACID SEQUENCING

(71) Applicants: Illumina Cambridge Limited, Cambridge (GB); Illumina Singapore Pte. Ltd., Singapore (SG); Illumina, Inc., San Diego, CA (US); Illumina Software, Inc., San Diego, CA (US)

(72) Inventors: Xiaolin Wu, Cambridge (GB); Patrick McCauley, Cambridge (GB); Madushani Dharmarwardana, Cambridge (GB); Saurabh Nirantar, Singapore (SG); Misha Golynskiy, San Diego, CA (US); Xiangyuan Yang, Singapore (SG); Ramesh Neelakandan, Singapore (SG); Benedict Mackworth, Cambridge (GB); Carole Anastasi, Cambridge (GB); Aathavan Karunakaran, San Diego, CA (US); Gery M. Vessere, San Diego, CA (US); David Bracher, San Diego, CA (US)

(21) Appl. No.: 18/325,610

(22) Filed: May 30, 2023

Related U.S. Application Data
(60) Provisional application No. 63/486,082, filed on Feb. 21, 2023.
(60) Provisional application No. 63/347,152, filed on May 31, 2022.

(51) Int. Cl. C12Q 1/6869; G01N 21/64

(52) U.S. Cl. CPC C12Q 1/6869; G01N 2021/6439; G01N 2021/6419; G01N 21/6428

(57) ABSTRACT

Embodiments of the present disclosure relate to kits, compositions, and methods for nucleic acid sequencing, for example, two-channel nucleic acid sequencing by synthesis using blue and green light excitation. In particular, unlabeled nucleotides for incorporation may be used in conjunction with affinity reagents containing detectable labels excitable by blue and/or green lights, for specific binding to each type of nucleotides incorporated.

### FIELD

The present disclosure generally relates to compositions, kits, methods and systems for nucleic acid sequencing applications.

### BACKGROUND

Nucleic acid sequencing methodology has evolved significantly from the chemical degradation methods used by Maxam and Gilbert and the strand elongation methods used by Sanger. Today several sequencing methodologies are in use which allow for the parallel processing of thousands of nucleic acids all in a single sequencing run. The instrumentation that performs such methods is typically large and expensive since the current methods typically rely on large amounts of expensive reagents and multiple sets of optic filters to record nucleic acid incorporation into sequencing reactions.

It has become clear that the need for high-throughput, smaller, less expensive DNA sequencing technologies will be beneficial for reaping the rewards of genome sequencing. Personalized healthcare is moving toward the forefront and will benefit from such technologies. The sequencing of an individual's genome to identify potential mutations and abnormalities will be crucial in identifying if a person has a particular disease, followed by subsequent therapies tailored to that individual. To accommodate such endeavor, sequencing technologies should not only have high throughput capabilities, but also have scalability. As such, there exists a need for new sequencing methods that improve on speed, error read, and are also cost effective.

### SUMMARY

The present disclosure provides next-generation sequencing kits, methods, systems and compositions. Certain disclosure relates to kits and compositions for two-channel nucleic acid sequencing applications using blue and green light excitation (e.g., lasers at 450-460 nm and 520-540 nm).

One aspect of the present disclosure relates to a kit for sequencing application, comprising:
a first type of unlabeled nucleotide comprising a first hapten;
a second type of unlabeled nucleotide comprising a second hapten;
a third type of unlabeled nucleotide; and
a set of affinity reagents comprising:
   a first affinity reagent comprising a first hapten-binding partner that is capable of specific binding to the first type of unlabeled nucleotide; and
   a second affinity reagent comprising a second hapten-binding partner that is capable of specific binding to the second type of unlabeled nucleotide;
   wherein the first affinity reagent comprises one or more first detectable labels that are excitable by a first excitation light source, the second affinity reagent comprises one or more second detectable labels that are excitable by a second excitation light source, and wherein the first detectable label is spectrally distinguishable from the second detectable label; and
wherein one of the first excitation light source and the second excitation light source has a wavelength of about 450 nm to about 460 nm, and the other one of the first excitation light source and the second excitation light source has a wavelength of about 520 nm to about 540 nm.

One aspect of the present disclosure relates to a protein assembly system, comprising:
a first protein labeled with one or more first detectable labels;
one or more hapten-containing linkers covalently attached to the first protein; and
one or more hapten-binding second proteins bound to one or more hapten-containing linkers;
wherein the one or more hapten-binding second proteins are labeled with one or more second detectable labels, and wherein the one or more first detectable labels are spectrally distinguishable from the one or more second detectable labels.

Another aspect of the present disclosure relates to a nucleotide conjugate comprising the protein assembly system described herein. In yet another aspect, the present disclosure relates to an oligonucleotide or polynucleotide comprising the nucleotide conjugate described herein.

A further aspect of the present disclosure relates to a labeled nanoparticle comprising: a polymer matrix comprising a plurality of detectable labels, wherein the backbone of the polymer comprises one or more cleavable moieties, and wherein the labeled nanoparticle is degradable into smaller polymeric chains upon cleavage of the cleavable moieties. In some embodiments, the labeled nanoparticle described herein may be used as detectable labels for the kit or the protein assembly system described herein. For example, additional embodiment of the present disclosure includes a labeled nanoparticle further comprising one or more proteins (e.g., antibody) attached thereto or coated thereon.

A further aspect of the present disclosure relates to use of the kit in nucleic acid sequencing. For example, the kit may be used in a method of determining the sequences of a plurality of different target polynucleotides, comprising:
(a) contacting a solid support with a solution comprising sequencing primers under hybridization conditions, wherein the solid support comprises a plurality of different target polynucleotides immobilized thereon; and the sequencing primers are complementary to at least a portion of the target polynucleotides;
(b) contacting the solid support with an aqueous solution comprising DNA polymerase and one or more of four different types of unlabeled nucleotides under conditions suitable for DNA polymerase-mediated primer extension, and incorporating one type of nucleotides into the sequencing primers to produce extended copy polynucleotides; wherein
   each of the four types of nucleotides comprises a 3′ blocking group;
   the first type of unlabeled nucleotide comprises a first hapten;
   the second type of unlabeled nucleotide comprises a second hapten;
(c) contacting the extended copy polynucleotides with a set of affinity reagents under conditions wherein one affinity reagent binds specifically to the incorporated unlabeled nucleotides to provide labeled extended copy polynucleotides;
(d) imaging the solid support and performing one or more fluorescent measurements of the extended copy polynucleotides; and
(e) removing the 3′ blocking group of the incorporated nucleotides;
wherein the set of affinity reagents comprises:
   a first affinity reagent comprising a first hapten-binding partner that is capable of specific binding to the first type of unlabeled nucleotides; and
   a second affinity reagent comprising a second hapten-binding partner that is capable of specific binding to the second type of unlabeled nucleotides;
   wherein the first affinity reagent comprises one or more first detectable labels that are excitable by a first excitation light source, the second affinity reagent comprises one or more second detectable labels that are excitable by a second excitation light source, and wherein the one or more first detectable labels are spectrally distinguishable from the one or more second detectable labels; and
   wherein one of the first excitation light source and the second excitation light source has a wavelength of about 450 nm to about 460 nm, and the other one of the first excitation light source and the second excitation light source has a wavelength of about 520 nm to about 540 nm.

In some embodiments of kits or sequencing methods described herein, the kits and sequencing method can reduce or eliminate sequence context effect or sequence specific effect, for example in two-channel sequencing by synthesis (SBS).

### BRIEF DESCRIPTION OF THE DRAWINGS

FIG. 1 schematically illustrates a dual-labeled protein assembly system according to an embodiment of the present disclosure.

FIG. 2A illustrates a standard non-depolymerizable fluorescent nanoparticle.

FIG. 2B illustrates a depolymerizable labeled nanoparticle according to an embodiment of the present disclosure.

FIG. 3A and FIG. 3B are scatterplots of a blue/green two-channel SBS run conducted with a set of unlabeled nucleotides and a two-tag affinity reagent mixture according to two different embodiments of the present disclosure.

FIG. 4A is a scatterplot of a blue/green two-channel SBS run conducted with a set of unlabeled nucleotides and a three-tag affinity reagent mixture according to an embodiment of the present disclosure.

FIG. 4B is a scatterplot of a blue/green two-channel SBS run with a universal ffN mix and a three-tag affinity reagent mixture where one of the affinity reagents is a dual-protein assembly system according to an embodiment of the present disclosure.

FIG. 5A and FIG. 5B are relative fluorescent intensity chart in solution, comparing ffA labeled with a standard coumarin dye C in comparison to affinity reagents labeled with coumarin dye I-1 and dye I-2 respectively.

FIGS. 6A-6C are scatterplot of blue/green two-channel MiSeq® SBS runs at cycle comparing a set of nucleotides comprising fully functionalized C nucleotide (ffC) labeled with coumarin dye C, to the nucleotide set comprising ffC labeled with dye I-1 or dye I-2.

FIG. 7A and FIG. 7B are bar charts illustrating the percentage of T Called signal remaining after 151 cycles of the standard two-channel MiSeq® SBS at increasing light dosage, comparing the standard coumarin dye C labeled ffC in the incorporation mixture to the ffC labeled with dye I-2 or dye I-4 respectively.

### DETAILED DESCRIPTION

### Definitions

Unless defined otherwise, all technical and scientific terms used herein have the same meaning as is commonly understood by one of ordinary skill in the art. The use of the term “including” as well as other forms, such as “include”, “includes,” and “included,” is not limiting. The use of the term “having” as well as other forms, such as “have”, “has,” and “had,” is not limiting. As used in this specification, whether in a transitional phrase or in the body of the claim, the terms “comprise(s)” and “comprising” are to be interpreted as having an open-ended meaning. That is, the above terms are to be interpreted synonymously with the phrases “having at least” or “including at least.” For example, when used in the context of a process, the term “comprising” means that the process includes at least the recited steps but may include additional steps. When used in the context of a compound, composition, or device, the term “comprising” means that the compound, composition, or device includes at least the recited features or components, but may also include additional features or components.

As used herein, common organic abbreviations are defined as follows:
° C. Temperature in degrees Centigrade
dATP Deoxyadenosine triphosphate
dCTP Deoxycytidine triphosphate
dGTP Deoxyguanosine triphosphate
dTTP Deoxythymidine triphosphate
ddNTP Dideoxynucleotide triphosphate
ffA Fully functionalized A nucleotide
ffC Fully functionalized C nucleotide
ffG Fully functionalized G nucleotide
ffN Fully functionalized nucleotide
ffT Fully functionalized T nucleotide
LED Light emitting diode
SBS Sequencing by synthesis

As used herein, the term “array” refers to a population of different probe molecules that are attached to one or more substrates such that the different probe molecules can be differentiated from each other according to relative location. An array can include different probe molecules that are each located at a different addressable location on a substrate. Alternatively, or additionally, an array can include separate substrates each bearing a different probe molecule, wherein the different probe molecules can be identified according to the locations of the substrates on a surface to which the substrates are attached or according to the locations of the substrates in a liquid. Exemplary arrays in which separate substrates are located on a surface include, without limitation, those including beads in wells as described, for example, in U.S. Pat. No. 6,355,431 B1, US 2002/0102578 and PCT Publication No. WO 00/63437. Exemplary formats that can be used in the invention to distinguish beads in a liquid array, for example, using a microfluidic device, such as a fluorescent activated cell sorter (FACS), are described, for example, in US Pat. No. 6,524,793. Further examples of arrays that can be used in the invention include, without limitation, those described in U.S. Pat Nos. 5,429,807; 5,436,327; 5,561,071; 5,583,211; 5,658,734; 5,837,858; 5,874,219; 5,919,523; 6,136,269; 6,287,768; 6,287,776; 6,288,220; 6,297,006; 6,291,193; 6,346,413; 6,416,949; 6,482,591; 6,514,751 and 6,610,482; and WO 93/17126; WO 95/11995; WO 95/35505; EP 742 287; and EP 799 897.

As used herein, the term “covalently attached” or “covalently bonded” refers to the forming of a chemical bonding that is characterized by the sharing of pairs of electrons between atoms. For example, a covalently attached polymer coating refers to a polymer coating that forms chemical bonds with a functionalized surface of a substrate, as compared to attachment to the surface via other means, for example, adhesion or electrostatic interaction. It will be appreciated that polymers that are attached covalently to a surface can also be bonded via means in addition to covalent attachment.

As used herein, the term “non-covalent interactions” differs from a covalent bond in that it does not involve the sharing of electrons, but rather involves more dispersed variations of electromagnetic interactions between molecules or within a molecule. Non-covalent interactions can be generally classified into four categories, electrostatic, π-effects, van der Waals forces, and hydrophobic effects. Non-limiting examples of electrostatic interactions include ionic interactions, hydrogen bonding (a specific type of dipole-dipole interaction), halogen bonding, etc. Van der Walls forces are a subset of electrostatic interaction involving permanent or induced dipoles or multipoles. π-effects can be broken down into numerous categories, including (but not limited to) ππinteractions, cation-π & anion-π interactions, and polar-π interactions. In general, π-effects are associated with the interactions of molecules with the π-orbitals of a molecular system, such as benzene. The hydrophobic effect is the tendency of nonpolar substances to aggregate in aqueous solution and exclude water molecules. Non-covalent interactions can be both intermolecular and intramolecular. Non-covalent interactions can be both intermolecular and intramolecular.

It is to be understood that certain radical naming conventions can include either a mono-radical or a di-radical, depending on the context. For example, where a substituent requires two points of attachment to the rest of the molecule, it is understood that the substituent is a di-radical. For example, a substituent identified as alkyl that requires two points of attachment includes di-radicals such as —CH₂—, —CH₂CH₂—, —CH₂CH(CH₃)CH₂—, and the like. Other radical naming conventions clearly indicate that the radical is a di-radical such as “alkylene” or “alkenylene.”

The term “halogen” or “halo,” as used herein, means any one of the radio-stable atoms of column 7 of the Periodic Table of the Elements, e.g., fluorine, chlorine, bromine, or iodine, with fluorine and chlorine being preferred.

As used herein, “Cₐ to C_{b},” “Cₐ-C_{b},” or “C_{a-b}” in which “a” and “b” are integers refer to the number of carbon atoms in an alkyl, alkenyl or alkynyl group, or the number of ring atoms of a cycloalkyl or aryl group. That is, the alkyl, the alkenyl, the alkynyl, the ring of the cycloalkyl, and ring of the aryl can contain from “a” to “b,” inclusive, carbon atoms. For example, a “C₁ to C₄ alkyl” group refers to all alkyl groups having from 1 to 4 carbons, that is, CH₃—, CH₃CH₂—, CH₃CH₂CH₂—, (CH₃)₂CH—, CH₃CH₂CH₂CH₂—, CH₃CH₂CH(CH₃)— and (CH₃)₃C—; a C₃ to C₄ cycloalkyl group refers to all cycloalkyl groups having from 3 to 4 carbon atoms, that is, cyclopropyl and cyclobutyl. Similarly, a “4 to 6 membered heterocyclyl” group refers to all heterocyclyl groups with 4 to 6 total ring atoms, for example, azetidine, oxetane, oxazoline, pyrrolidine, piperidine, piperazine, morpholine, and the like. If no “a” and “b” are designated with regard to an alkyl, alkenyl, alkynyl, cycloalkyl, or aryl group, the broadest range described in these definitions is to be assumed. As used herein, the term “C₁-C₆” includes C₁, C₂, C₃, C₄, C₅ and C₆, and a range defined by any of the two numbers. For example, C₁-C₆ alkyl includes C₁, C₂, C₃, C₄, C₅ and C₆ alkyl, C₂-C₆ alkyl, C₁-C₃ alkyl, etc. Similarly, C₂-C₆ alkenyl includes C₂, C₃, C₄, C₅ and C₆ alkenyl, C₂-C₅ alkenyl, C₃ -C₄ alkenyl, etc.; and C₂-C₆ alkynyl includes C₂, C₃, C₄, C₅ and C₆ alkynyl, C₂-C₅ alkynyl, C₃-C₄ alkynyl, etc. C₃-C₈ cycloalkyl each includes hydrocarbon ring containing 3, 4, 5, 6, 7 and 8 carbon atoms, or a range defined by any of the two numbers, such as C₃-C₇ cycloalkyl or C₅-C₆ cycloalkyl.

As used herein, “alkyl” refers to a straight or branched hydrocarbon chain that is fully saturated (i.e., contains no double or triple bonds). The alkyl group may have 1 to 20 carbon atoms (whenever it appears herein, a numerical range such as “1 to 20” refers to each integer in the given range; e.g., “1 to 20 carbon atoms” means that the alkyl group may consist of 1 carbon atom, 2 carbon atoms, 3 carbon atoms, etc., up to and including 20 carbon atoms, although the present definition also covers the occurrence of the term “alkyl” where no numerical range is designated). The alkyl group may also be a medium size alkyl having 1 to 9 carbon atoms. The alkyl group could also be a lower alkyl having 1 to 6 carbon atoms. The alkyl group may be designated as “C₁-C₄ alkyl” or similar designations. By way of example only, “C₁-C₆ alkyl” indicates that there are one to six carbon atoms in the alkyl chain, i.e., the alkyl chain is selected from the group consisting of methyl, ethyl, propyl, iso-propyl, n-butyl, iso-butyl, sec-butyl, and t-butyl. Typical alkyl groups include, but are in no way limited to, methyl, ethyl, propyl, isopropyl, butyl, isobutyl, tertiary butyl, pentyl, hexyl, and the like.

As used herein, “alkoxy” refers to the formula —OR wherein R is an alkyl as is defined above, such as “C₁-C₉ alkoxy,” including but not limited to methoxy, ethoxy, n-propoxy, 1-methylethoxy (isopropoxy), n-butoxy, iso-butoxy, sec-butoxy, and tert-butoxy, and the like.

As used herein, “alkenyl” refers to a straight or branched hydrocarbon chain containing one or more double bonds. The alkenyl group may have 2 to 20 carbon atoms, although the present definition also covers the occurrence of the term “alkenyl” where no numerical range is designated. The alkenyl group may also be a medium size alkenyl having 2 to 9 carbon atoms. The alkenyl group could also be a lower alkenyl having 2 to 6 carbon atoms. The alkenyl group may be designated as “C₂-C₆ alkenyl” or similar designations. By way of example only, “C₂-C₆ alkenyl” indicates that there are two to six carbon atoms in the alkenyl chain, i.e., the alkenyl chain is selected from the group consisting of ethenyl, propen-1-yl, propen-2-yl, propen-3-yl, buten- 1-yl, buten-2-yl, buten-3-yl, buten-4-yl, 1-methyl-propen-1-yl, 2-methyl-propen-1-yl, 1-ethyl-ethen-1-yl, 2-methyl-propen-3-yl, buta-1,3-dienyl, buta-1,2,-dienyl, and buta-1,2-dien-4-yl. Typical alkenyl groups include, but are in no way limited to, ethenyl, propenyl, butenyl, pentenyl, and hexenyl, and the like.

The term “aromatic” refers to a ring or ring system having a conjugated pi electron system and includes both carbocyclic aromatic (e.g., phenyl) and heterocyclic aromatic groups (e.g., pyridine). The term includes monocyclic or fused-ring polycyclic (i.e., rings which share adjacent pairs of atoms) groups provided that the entire ring system is aromatic.

As used herein, “aryl” refers to an aromatic ring or ring system (i.e., two or more fused rings that share two adjacent carbon atoms) containing only carbon in the ring backbone. When the aryl is a ring system, every ring in the system is aromatic. The aryl group may have 6 to 18 carbon atoms, although the present definition also covers the occurrence of the term “aryl” where no numerical range is designated. In some embodiments, the aryl group has 6 to 10 carbon atoms. The aryl group may be designated as “C₆-C₁₀ aryl,” “C₆ or C₁₀ aryl,” or similar designations. Examples of aryl groups include, but are not limited to, phenyl, naphthyl, azulenyl, and anthracenyl.

An “aralkyl” or “arylalkyl” is an aryl group connected, as a substituent, via an alkylene group, such as “C₇₋₁₄ aralkyl” and the like, including but not limited to benzyl, 2-phenylethyl, 3-phenylpropyl, and naphthylalkyl. In some cases, the alkylene group is a lower alkylene group (i.e., a C₁-C₆ alkylene group).

As used herein, “aryloxy” refers to RO— in which R is an aryl, as defined above, such as but not limited to phenyl.

As used herein, “heteroaryl” refers to an aromatic ring or ring system (i.e., two or more fused rings that share two adjacent atoms) that contain(s) one or more heteroatoms, that is, an element other than carbon, including but not limited to, nitrogen, oxygen and sulfur, in the ring backbone. When the heteroaryl is a ring system, every ring in the system is aromatic. The heteroaryl group may have 5-18 ring members (i.e., the number of atoms making up the ring backbone, including carbon atoms and heteroatoms), although the present definition also covers the occurrence of the term “heteroaryl” where no numerical range is designated. In some embodiments, the heteroaryl group has 5 to 10 ring members or 5 to 7 ring members. The heteroaryl group may be designated as “5-7 membered heteroaryl,” “5-10 membered heteroaryl,” or similar designations. Examples of heteroaryl rings include, but are not limited to, furyl, thienyl, phthalazinyl, pyrrolyl, oxazolyl, thiazolyl, imidazolyl, pyrazolyl, isoxazolyl, isothiazolyl, triazolyl, thiadiazolyl, pyridinyl, pyridazinyl, pyrimidinyl, pyrazinyl, triazinyl, quinolinyl, isoquinolinyl, benzoimidazolyl, benzoxazolyl, benzothiazolyl, indolyl, isoindolyl, and benzothienyl.

A “heteroaralkyl” or “heteroarylalkyl” is heteroaryl group connected, as a substituent, via an alkylene group. Examples include but are not limited to 2-thienylmethyl, 3-thienylmethyl, furylmethyl, thienylethyl, pyrrolylalkyl, pyridylalkyl, isoxazollylalkyl, and imidazolylalkyl. In some cases, the alkylene group is a lower alkylene group (i.e., a C₁-C₆ alkylene group).

As used herein, “carbocyclyl” means a non-aromatic cyclic ring or ring system containing only carbon atoms in the ring system backbone. When the carbocyclyl is a ring system, two or more rings may be joined together in a fused, bridged or spiro-connected fashion. Carbocyclyls may have any degree of saturation provided that at least one ring in a ring system is not aromatic. Thus, carbocyclyls include cycloalkyls, cycloalkenyls, and cycloalkynyls. The carbocyclyl group may have 3 to 20 carbon atoms, although the present definition also covers the occurrence of the term “carbocyclyl” where no numerical range is designated. The carbocyclyl group may also be a medium size carbocyclyl having 3 to 10 carbon atoms. The carbocyclyl group could also be a carbocyclyl having 3 to 6 carbon atoms. The carbocyclyl group may be designated as “C₃-C₆ carbocyclyl” or similar designations. Examples of carbocyclyl rings include, but are not limited to, cyclopropyl, cyclobutyl, cyclopentyl, cyclohexyl, cyclohexenyl, 2,3-dihydro-indene, bicycle [2.2.2]octanyl, adamantyl, and spiro[4.4]nonanyl.

As used herein, “cycloalkyl” means a fully saturated carbocyclyl ring or ring system. Examples include cyclopropyl, cyclobutyl, cyclopentyl, and cyclohexyl.

As used herein, “heterocyclyl” means a non-aromatic cyclic ring or ring system containing at least one heteroatom in the ring backbone. Heterocyclyls may be joined together in a fused, bridged or spiro-connected fashion. Heterocyclyls may have any degree of saturation provided that at least one ring in the ring system is not aromatic. The heteroatom(s) may be present in either a non-aromatic or aromatic ring in the ring system. The heterocyclyl group may have 3 to 20 ring members (i.e., the number of atoms making up the ring backbone, including carbon atoms and heteroatoms), although the present definition also covers the occurrence of the term “heterocyclyl” where no numerical range is designated. The heterocyclyl group may also be a medium size heterocyclyl having 3 to 10 ring members. The heterocyclyl group could also be a heterocyclyl having 3 to 6 ring members. The heterocyclyl group may be designated as “3-6 membered heterocyclyl” or similar designations. In preferred six membered monocyclic heterocyclyls, the heteroatom(s) are selected from one up to three of O, N or S, and in preferred five membered monocyclic heterocyclyls, the heteroatom(s) are selected from one or two heteroatoms selected from O, N, or S. Examples of heterocyclyl rings include, but are not limited to, azepinyl, acridinyl, carbazolyl, cinnolinyl, dioxolanyl, imidazolinyl, imidazolidinyl, morpholinyl, oxiranyl, oxepanyl, thiepanyl, piperidinyl, piperazinyl, dioxopiperazinyl, pyrrolidinyl, pyrrolidonyl, pyrrolidionyl, 4-piperidonyl, pyrazolinyl, pyrazolidinyl, 1,3-dioxinyl, 1,3-dioxanyl, 1,4-dioxinyl, 1,4-dioxanyl, 1,3-oxathianyl, 1,4-oxathiinyl, 1,4-oxathianyl, 2H-1,2-oxazinyl, trioxanyl, hexahydro-1,3,5-triazinyl, 1,3-dioxolyl, 1,3-dioxolanyl, 1,3-dithiolyl, 1,3-dithiolanyl, isoxazolinyl, isoxazolidinyl, oxazolinyl, oxazolidinyl, oxazolidinonyl, thiazolinyl, thiazolidinyl, 1,3-oxathiolanyl, indolinyl, is oindolinyl, tetrahydrofuranyl, tetrahydropyranyl, tetrahydrothiophenyl, tetrahydrothiopyranyl, tetrahydro-1,4-thiazinyl, thiamorpholinyl, dihydrobenzofuranyl, benzimidazolidinyl, and tetrahydroquinoline.

As used herein, “(aryl)alkyl” refer to an aryl group, as defined above, connected, as a substituent, via an alkylene group, as described above. The alkylene and aryl group of an aralkyl may be substituted or unsubstituted. Examples include but are not limited to benzyl, 2-phenylalkyl, 3-phenylalkyl, and naphthylalkyl. In some embodiments, the alkylene is an unsubstituted straight chain containing 1, 2, 3, 4, 5, or 6 methylene unit(s).

As used herein, “(heteroaryl)alkyl” refer to a heteroaryl group, as defined above, connected, as a substituent, via an alkylene group, as defined above. The alkylene and heteroaryl group of heteroaralkyl may be substituted or unsubstituted. Examples include but are not limited to 2-thienylalkyl, 3-thienylalkyl, furylalkyl, thienylalkyl, pyrrolylalkyl, pyridylalkyl, isoxazolylalkyl, and imidazolylalkyl, and their benzo-fused analogs. In some embodiments, the alkylene is an unsubstituted straight chain containing 1, 2, 3, 4, 5, or 6 methylene unit(s).

As used herein, “(heterocyclyl)alkyl” refer to a heterocyclic or a heterocyclyl group, as defined above, connected, as a substituent, via an alkylene group, as defined above. The alkylene and heterocyclyl groups of a (heterocyclyl)alkyl may be substituted or unsubstituted. Examples include but are not limited to (tetrahydro-2H-pyran-4-yl)methyl, (piperidin-4-yl)ethyl, (piperidin-4-yl)propyl, (tetrahydro-2H-thiopyran-4-yl)methyl, and (1,3-thiazinan-4-yl)methyl. In some embodiments, the alkylene is an unsubstituted straight chain containing 1, 2, 3, 4, 5, or 6 methylene unit(s).

As used herein, “(carbocyclyl)alkyl” refer to a carbocyclyl group (as defined herein) connected, as a substituent, via an alkylene group. Examples include but are not limited to cyclopropylmethyl, cyclobutylmethyl, cyclopentylethyl, and cyclohexylpropyl. In some embodiments, the alkylene is an unsubstituted straight chain containing 1, 2, 3, 4, 5, or 6 methylene unit(s).

As used herein, “alkoxyalkyl” or “(alkoxy)alkyl” refers to an alkoxy group connected via an alkylene group, such as C₂-C₈alkoxyalkyl, or (C₁-C₆ alkoxy)C₁-C₆alkyl, for example, —(CH₂)₁₋₃—OCH₃.

As used herein, “—O-alkoxyalkyl” or “—O-(alkoxy)alkyl” refers to an alkoxy group connected via an —O-(alkylene) group, such as —O-(C₁-C₆ alkoxy)C₁-C₆ alkyl, for example, —O—(CH₂)₁₋₃—OCH₃.

As used herein, “haloalkyl” refers to an alkyl group in which one or more of the hydrogen atoms are replaced by a halogen (e.g., mono-haloalkyl, di-haloalkyl, and tri-haloalkyl). Such groups include but are not limited to, chloromethyl, fluoromethyl, difluoromethyl, trifluoromethyl and 1-chloro-2-fluoromethyl, 2-fluoroisobutyl. A haloalkyl may be substituted or unsubstituted.

As used herein, “haloalkoxy” refers to an alkoxy group in which one or more of the hydrogen atoms are replaced by a halogen (e.g., mono-haloalkoxy, di-haloalkoxy and tri- haloalkoxy). Such groups include but are not limited to, chloromethoxy, fluoromethoxy, difluoromethoxy, trifluoromethoxy and 1-chloro-2-fluoromethoxy, 2-fluoroisobutoxy. A haloalkoxy may be substituted or unsubstituted.

An “amino” group refers to a —NH₂ group. The term “mono-substituted amino group” as used herein refers to an amino (—NH₂) group where one of the hydrogen atom is replaced by a substituent. The term “di-substituted amino group” as used herein refers to an amino (—NH₂) group where each of the two hydrogen atoms is replaced by a substituent. The term “optionally substituted amino,” as used herein refer to a —NR_{A}R_{B} group where R_{A} and R_{B} are independently hydrogen, alkyl, cycloalkyl, aryl, heteroaryl, heterocyclyl, aralkyl, or heterocyclyl(alkyl), as defined herein.

An “O-carboxy” group refers to a “—OC(═O)R” group in which R is selected from hydrogen, C₁-C₆ alkyl, C₂-C₆ alkenyl, C₂-C₆ alkynyl, C₃-C₇ carbocyclyl, C₆-C₁₀ aryl, 5-10 membered heteroaryl, and 3-10 membered heterocyclyl, as defined herein.

A “C-carboxy” group refers to a “—C(═O)OR” group in which R is selected from the group consisting of hydrogen, C₁-C₆ alkyl, C₂-C₆ alkenyl, C₂-C₆ alkynyl, C₃-C₇ carbocyclyl, C₆-C₁₀ aryl, 5-10 membered heteroaryl, and 3-10 membered heterocyclyl, as defined herein. A non-limiting example includes carboxyl (i.e., —C(═O)OH).

A “sulfonyl” group refers to an “—SO₂R” group in which R is selected from hydrogen, C₁-C₆ alkyl, C₂-C₆ alkenyl, C₂-C₆ alkynyl, C₃-C₇ carbocyclyl, C₆-C₁₀ aryl, 5-10 membered heteroaryl, and 3-10 membered heterocyclyl, as defined herein.

A “sulfino” group refers to a “—S(═O)OH” group.

A “sulfo” group refers to a “—S(═O)₂OH” or “—SO₃H” group.

A “sulfonate” group refers to a “—SO₃⁻” group.

A “sulfate” group refers to “—SO₄⁻” group.

A “S-sulfonamido” group refers to a “—SO₂NR_{A}R_{B}” group in which R_{A} and R_{B} are each independently selected from hydrogen, C₁-C₆ alkyl, C₂-C₆ alkenyl, C₂-C₆ alkynyl, C₃-C₇ carbocyclyl, C₆-C₁₀ aryl, 5-10 membered heteroaryl, and 3-10 membered heterocyclyl, as defined herein.

An “N-sulfonamido” group refers to a “—N(R_{A})SO₂R_{B}” group in which R_{A} and R_{b} are each independently selected from hydrogen, C₁-C₆ alkyl, C₂-C₆ alkenyl, C₂-C₆ alkynyl, C₃-C₇ carbocyclyl, C₆-C₁₀ aryl, 5-10 membered heteroaryl, and 3-10 membered heterocyclyl, as defined herein.

A “C-amido” group refers to a “—C(═O)NR_{A}R_{B}” group in which R_{A} and R_{B} are each independently selected from hydrogen, C₁-C₆ alkyl, C₂-C₆ alkenyl, C₂-C₆ alkynyl, C₃-C₇ carbocyclyl, C₆-C₁₀ aryl, 5-10 membered heteroaryl, and 3-10 membered heterocyclyl, as defined herein.

An “N-amido” group refers to a “—N(R_{A})C(═O)R_{B}” group in which R_{A} and R_{B} are each independently selected from hydrogen, C₁-C₆ alkyl, C₂-C₆ alkenyl, C₂-C₆ alkynyl, C₃-C₇ carbocyclyl, C₆-C₁₀ aryl, 5-10 membered heteroaryl, and 3-10 membered heterocyclyl, as defined herein.

An “O-carbamyl” group refers to a “—OC(═O)N(R_{A}R_{B})” group in which R_{A} and R_{B} can be the same as defined with respect to S-sulfonamido. An O-carbamyl may be substituted or unsubstituted.

An “N-carbamyl” group refers to an “ROC(═O)N(R_{A})—” group in which R and R_{A} can be the same as defined with respect to N-sulfonamido. An N-carbamyl may be substituted or unsubstituted.

An “O -thiocarbamyl” group refers to a “—OC(═S)—N(R_{A}R_{B})” group in which R_{A} and R_{B} can be the same as defined with respect to S-sulfonamido. An O-thiocarbamyl may be substituted or unsubstituted.

An “N-thiocarbamyl” group refers to an “ROC(═S)N(R_{A})—” group in which R and R_{A} can be the same as defined with respect to N-sulfonamido. An N-thiocarbamyl may be substituted or unsubstituted.

The term “alkylamino” or “(alkyl)amino” refers to an amino group wherein one or both hydrogen is replaced by an alkyl group.

An “(alkoxy)alkyl” group refers to an alkoxy group connected via an alkylene group, such as a “(C₁-C₆ alkoxy) C₁-C₆ alkyl” and the like.

The term “hydroxy” as used herein refers to a —OH group.

The term “cyano” group as used herein refers to a “—CN” group.

The term “azido” as used herein refers to a —N₃ group.

The term “isonitrile” as used herein refers to a “—N⁺≡C⁻” group.

When a group is described as “optionally substituted” it may be either unsubstituted or substituted. Likewise, when a group is described as being “substituted,” the substituent may be selected from one or more of the indicated substituents. As used herein, a substituted group is derived from the unsubstituted parent group in which there has been an exchange of one or more hydrogen atoms for another atom or group. Unless otherwise indicated, when a group is deemed to be “substituted,” it is meant that the group is substituted with one or more substituents independently selected from C₁-C₆ alkyl, C₁-C₆ alkenyl, C₁-C₆ alkynyl, C₁-C₆ heteroalkyl, C₃-C₇ carbocyclyl (optionally substituted with halo, C₁-C₆ alkyl, C₁-C₆ alkoxy, C₁-C₆ haloalkyl, and C₁-C₆ haloalkoxy), C₃-C₇ carbocyclyl-C₁-C₆-alkyl (optionally substituted with halo, C₁-C₆ alkyl, C₁-C₆ alkoxy, C₁-C₆ haloalkyl, and C₁-C₆ haloalkoxy), 3-10 membered heterocyclyl (optionally substituted with halo, C₁-C₆ alkyl, C₁-C₆ alkoxy, C₁-C₆ haloalkyl, and C₁-C₆ haloalkoxy), 3-10 membered heterocyclyl-C₁-C₆-alkyl (optionally substituted with halo, C₁-C₆ alkyl, C₁-C₆ alkoxy, C₁-C₆ haloalkyl, and C₁-C₆ haloalkoxy), aryl (optionally substituted with halo, C₁-C₆ alkyl, C₁-C₆ alkoxy, C₁-C₆ haloalkyl, and

C₁-C₆ haloalkoxy), (aryl)C₁-C₆ alkyl (optionally substituted with halo, C₁-C₆ alkyl, C₁-C₆ alkoxy, C₁-C₆ haloalkyl, and C₁-C₆ haloalkoxy), 5-10 membered heteroaryl (optionally substituted with halo, C₁-C₆ alkyl, C₁-C₆ alkoxy, C₁-C₆ haloalkyl, and C₁-C₆ haloalkoxy), (5-10 membered heteroaryl)C₁-C₆ alkyl (optionally substituted with halo, C₁-C₆ alkyl, C₁-C₆ alkoxy, C₁-C₆ haloalkyl, and C₁-C₆ haloalkoxy), halo, —CN, hydroxy, C₁-C₆ alkoxy, (C₁-C₆ alkoxy)C₁-C₆ alkyl, —O(C₁-C₆ alkoxy)C₁-C₆ alkyl; (C₁-C₆ haloalkoxy)C₁-C₆ alkyl; —O(C₁-C₆ haloalkoxy)C₁-C₆ alkyl; aryloxy, sulfhydryl (mercapto), halo(C₁-C₆)alkyl (e.g., —CF₃), halo(C₁-C₆)alkoxy (e.g., —OCF₃), C₁-C₆ alkylthio, arylthio, amino, amino(C₁-C₆)alkyl, nitro, O-carbamyl, N-carbamyl, O-thiocarbamyl, N-thiocarbamyl, C-amido, N-amido, S-sulfonamido, N-sulfonamido, C-carboxy, O-carboxy, acyl, cyanato, isocyanato, thiocyanato, isothiocyanato, sulfinyl, sulfonyl, —SO₃H, sulfonate, sulfate, sulfino, —OSO₂C₁₋₄alkyl, monophosphate, diphosphate, triphosphate, and oxo (═O). Wherever a group is described as “optionally substituted” that group can be substituted with the above substituents.

In each instance where a single mesomeric form of a compound described herein is shown, the alternative mesomeric forms are equally contemplated.

As used herein, a “nucleotide” includes a nitrogen containing heterocyclic base, a sugar, and one or more phosphate groups. They are monomeric units of a nucleic acid sequence. In RNA, the sugar is a ribose, and in DNA a deoxyribose, i.e., a sugar lacking a hydroxyl group that is present in ribose. The nitrogen containing heterocyclic base can be purine, deazapurine, or pyrimidine base. Purine bases include adenine (A) and guanine (G), and modified derivatives or analogs thereof, such as 7-deaza adenine or 7-deaza guanine. Pyrimidine bases include cytosine (C), thymine (T), and uracil (U), and modified derivatives or analogs thereof. The C-1 atom of deoxyribose is bonded to N-1 of a pyrimidine or N-9 of a purine.

As used herein, a “nucleotide conjugate” generally refers to a nucleotide labeled with a fluorescent moiety, optionally through a cleavage linker as described herein. In some embodiment, when a nucleotide conjugate is described as an unlabeled nucleotide, such nucleotide does not include a fluorescent moiety. In some further embodiments, an unlabeled nucleotide conjugate also does not have a cleavable linker.

As used herein, a “nucleoside” is structurally similar to a nucleotide but is missing the phosphate moieties. An example of a nucleoside analogue would be one in which the label is linked to the base and there is no phosphate group attached to the sugar molecule. The term “nucleoside” is used herein in its ordinary sense as understood by those skilled in the art. Examples include, but are not limited to, a ribonucleoside comprising a ribose moiety and a deoxyribonucleoside comprising a deoxyribose moiety. A modified pentose moiety is a pentose moiety in which an oxygen atom has been replaced with a carbon and/or a carbon has been replaced with a sulfur or an oxygen atom. A “nucleoside” is a monomer that can have a substituted base and/or sugar moiety. Additionally, a nucleoside can be incorporated into larger DNA and/or RNA polymers and oligomers.

The term “purine base” is used herein in its ordinary sense as understood by those skilled in the art and includes its tautomers. Similarly, the term “pyrimidine base” is used herein in its ordinary sense as understood by those skilled in the art and includes its tautomers. A non-limiting list of optionally substituted purine-bases includes purine, adenine, guanine, deazapurine, 7-deaza adenine, 7-deaza guanine. hypoxanthine, xanthine, alloxanthine, 7-alkylguanine (e.g., 7-methylguanine), theobromine, caffeine, uric acid and isoguanine. Examples of pyrimidine bases include, but are not limited to, cytosine, thymine, uracil, 5,6-dihydrouracil and 5-alkylcytosine (e.g., 5-methylcytosine).

As used herein, when an oligonucleotide or polynucleotide is described as “comprising” a nucleoside or nucleotide described herein, it means that the nucleoside or nucleotide described herein forms a covalent bond with the oligonucleotide or polynucleotide. Similarly, when a nucleoside or nucleotide is described as part of an oligonucleotide or polynucleotide, such as “incorporated into” an oligonucleotide or polynucleotide, it means that the nucleoside or nucleotide described herein forms a covalent bond with the oligonucleotide or polynucleotide. In some such embodiments, the covalent bond is formed between a 3′ hydroxy group of the oligonucleotide or polynucleotide with the 5′ phosphate group of a nucleotide described herein as a phosphodiester bond between the 3′ carbon atom of the oligonucleotide or polynucleotide and the 5′ carbon atom of the nucleotide.

As used herein, the term “cleavable linker” is not meant to imply that the whole linker is required to be removed. The cleavage site can be located at a position on the linker that ensures that part of the linker remains attached to the detectable label and/or nucleoside or nucleotide moiety after cleavage.

As used herein, “derivative” or “analog” means a synthetic nucleotide or nucleoside derivative having modified base moieties and/or modified sugar moieties. Such derivatives and analogs are discussed in, e.g., Scheit, Nucleotide Analogs (John Wiley & Son, 1980) and Uhlman et al., Chemical Reviews 90:543-584, 1990. Nucleotide analogs can also comprise modified phosphodiester linkages, including phosphorothioate, phosphorodithioate, alkyl-phosphonate, and phosphoramidate linkages. “Derivative,” “analog” and “modified” as used herein, may be used interchangeably, and are encompassed by the terms “nucleotide” and “nucleoside” defined herein.

As used herein, a biotin moiety-containing molecule or an analog thereof comprises the biotin moiety of structure

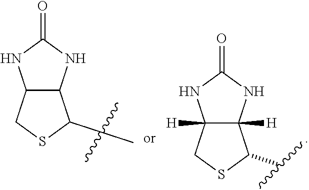 [SMILES: *C1SCC2NC(=O)NC21 ; *[C@@H]1SC[C@@H]2NC(=O)N[C@@H]21]

In some cases, biotin moiety is attached to the remaining portion of the molecule via a linker, such as

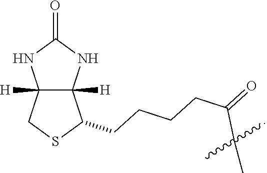 [SMILES: *C(=O)CCCC[C@@H]1SC[C@@H]2NC(=O)N[C@@H]21]

The analog of the biotin moiety-containing molecule may include a substituted biotin moiety.

As used herein, an alkyl chloride-containing molecule or an analog thereof comprises the structure

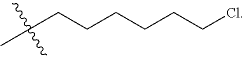 [SMILES: *CCCCCCCl]

The analog of the alkyl chloride moiety-containing molecule may include a substituted alkyl chloride moiety.

As used herein, a dinitrophenyl (DNP) moiety-containing molecule or an analog thereof comprises the structure

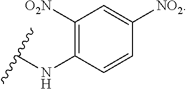 [SMILES: *Nc1ccc([N+](=O)[O-])cc1[N+](=O)[O-]]

In some cases, DNP moiety is attached to the
remaining portion of the molecule via a linker, such as

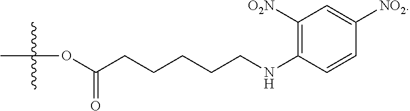 [SMILES: *OC(=O)CCCCCNc1ccc([N+](=O)[O-])cc1[N+](=O)[O-]]

The analog of the DNP moiety-containing molecule may include a substituted DNP moiety.

As used herein, a digoxigenin (DIG) moiety-containing molecule or an analog thereof comprises the structure

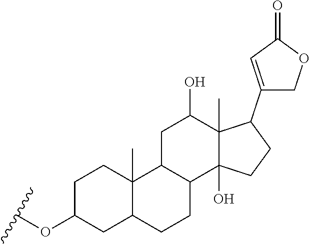 [SMILES: *OC1CCC2(C)C(CCC3C2CC(O)C2(C)C(C4=CC(=O)OC4)CCC32O)C1]

or diastereomers thereof such as

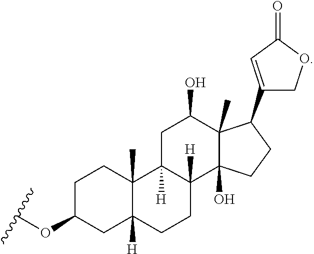

In some cases, DIG moiety is attached to the remining portion of the molecule via a linker, such as

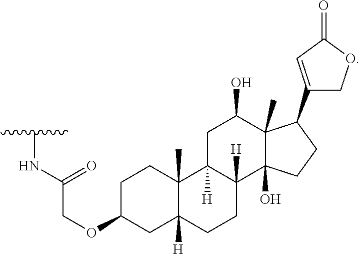

The analog of the DIG moiety-containing molecule may include a substituted DIG moiety.

As used herein, a β-N-acetylglucosamine (O-GlcNAc) moiety-containing molecule or an analog thereof comprises the structure

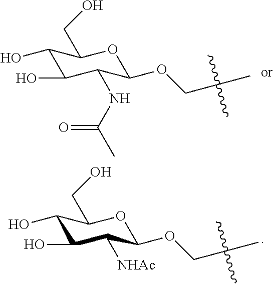

The analog of the O-GlcNAc moiety-containing molecule may include a substituted O-GlcNAc moiety.

As used herein, an alkyl guanine moiety-containing molecule or an analog thereof comprises the structure

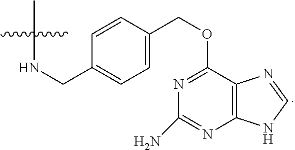 [SMILES: *NCc1ccc(COc2nc(N)nc3[nH]cnc23)cc1]

The analog of the alkyl guanine moiety-containing molecule may include a substituted alkyl guanine moiety.

As used herein, a 3-nitrotyrosine moiety-containing molecule or an analog thereof comprises the structure

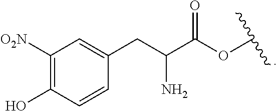 [SMILES: *OC(=O)C(N)Cc1ccc(O)c([N+](=O)[O-])c1]

The analog of the 3-nitrotyrosine moiety containing molecule may include a substituted 3-nitrotyrosine moiety.

As used herein, anti-DNP antibody refers to an antibody capable of specific binding to a DNP moiety described herein.

As used herein, anti-DIG antibody refers to an antibody capable of specific binding to a DIG moiety described herein.

As used herein, wheat germ agglutinin (WGA) refers to a lectin capable of binding O-GlcNAc moiety described herein.

As used herein, SNAP-Tag® refers to a commercially available protein tag. SNAP-Tag® is capable of specific binding to an alkyl guanine moiety described herein.

As used herein, HaloTag® refers to a commercially available protein tag. HaloTag® is capable of specific binding to alkyl chloride moiety described herein.

As used herein, anti-nitrotyrosine antibody refers to an antibody capable of specific binding to a 3-nitrotyrosine moiety described herein.

As used herein, the term “phosphate” is used in its ordinary sense as understood by those skilled in the art, and includes its protonated forms (for example,

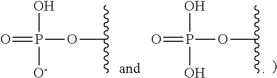 [SMILES: *OP(=O)([O-])O ; *OP(=O)(O)O]

As used herein, the terms “monophosphate,” “diphosphate,” and “triphosphate” are used in their ordinary sense as understood by those skilled in the art and include protonated forms.

As understood by one of ordinary skill in the art, a compound such as a nucleotide conjugate described herein may exist in ionized form, e.g., containing a —CO₂⁻, —SO₃⁻ or —O⁻. If a compound contains a positively or negatively charged substituent group, it may also contain a negatively or positively charged counterion such that the compound as a whole is neutral. In other aspects, the compound may exist in a salt form, where the counterion is provided by a conjugate acid or base.

As used herein, the term “phasing” refers to a phenomenon in SBS that is caused by incomplete removal of the 3′ terminators and fluorophores, and/or failure to complete the incorporation of a portion of DNA strands within clusters by polymerases at a given sequencing cycle. Prephasing is caused by the incorporation of nucleotides without effective 3′ terminators, wherein the incorporation event goes 1 cycle ahead due to a termination failure. Phasing and prephasing cause the measured signal intensities for a specific cycle to consist of the signal from the current cycle as well as noise from the preceding and following cycles. As the number of cycles increases, the fraction of sequences per cluster affected by phasing and prephasing increases, hampering the identification of the correct base. Prephasing can be caused by the presence of a trace amount of unprotected or unblocked 3′-OH nucleotides during sequencing by synthesis (SBS). The unprotected 3′-OH nucleotides could be generated during the manufacturing processes or possibly during the storage and reagent handling processes.

As used herein, the term “sequence context effect” or “sequence specific effect” refers to the effect that the intensity of a base as shown in a cloud scatterplot may be impacted by the preceding sequence context during sequencing by synthesis (SBS), in particular the two-channel SBS. This intensity modulation adds noise to the system and can cause miscalls when the sequence-specific intensity modulation shift a given cluster's intensity towards a decision boundary. It is also known as sequence specific errors (SSE). Without being bound by a particular theory, one reason for the sequence-specific intensity shifts is differential incorporation of fully functionalized nucleotides (ffNs) labeled with different dyes (e.g., green ffA and blue ffA). Another reason for causing fluorescent signal intensity shirt may be due to the preceding bases show different effect on the incorporated labeled nucleotides (e.g., quenching or enhancing dye signal intensity). As described in the present disclosure, using a single fully functionalized nucleotide (ffN) in connection with affinity reagent(s) that can produce color in two channels can reduce or eliminate sequence-specific intensity shifts and thereby improve base calling accuracy.

### Kits

One aspect of the present disclosure relates to a kit for sequencing application, comprising:
a first type of unlabeled nucleotide comprising a first hapten;
a second type of unlabeled nucleotide comprising a second hapten;
a third type of unlabeled nucleotide; and
a set of affinity reagents comprising:
a first affinity reagent comprising a first hapten-binding partner that is capable of specific binding to the first type of unlabeled nucleotide; and
a second affinity reagent comprising a second hapten-binding partner that is capable of specific binding to the second type of unlabeled nucleotide;
wherein the first affinity reagent comprises one or more first detectable labels that are excitable by a first excitation light source, the second affinity reagent comprises one or more second detectable labels that are excitable by a second excitation light source, and wherein the first detectable label is spectrally distinguishable from the second detectable label; and
wherein one of the first excitation light source and the second excitation light source has a wavelength of about 450 nm to about 460 nm, and the other one of the first excitation light source and the second excitation light source has a wavelength of about 520 nm to about 540 nm.

In some embodiments of the kit described herein, the first hapten is covalently attached to the nucleobase of the first type of unlabeled nucleotide via a cleavable linker.

In some embodiments of the kit described herein, the second hapten is covalently attached to the nucleobase of the second type of unlabeled nucleotide via a cleavable linker.

### Two-Tag System

In some embodiments of the kit described herein, the third type of unlabeled nucleotide comprises a mixture of the third type of unlabeled nucleotide comprising the first hapten and the third type of unlabeled nucleotide comprising the second hapten, and wherein both the first affinity reagent and the second affinity reagent are capable of specific binding to the third type of unlabeled nucleotide. In a further embodiment, each of the first hapten and the second hapten is covalently attached to the nucleobase of the third type of nucleotide via a cleavable linker. In a further embodiment, the first hapten comprises a biotin moiety and the first hapten-binding partner comprises an avidin (e.g., streptavidin, neutravidin, flavidin, etc.). In a further embodiment, the avidin is streptavidin. In a further embodiment, the avidin is neutravidin. In a further embodiment, one or more biotin binding sites of the avidin are blocked by a biotin moiety-containing molecule or an analog thereof. In a further embodiment, all remaining biotin binding sites of the avidin are blocked by the biotin moiety-containing molecule or an analog thereof. In a further embodiment, the second hapten comprises a dinitrophenyl (DNP) moiety and the second hapten-binding partner comprises an anti-DNP antibody. In some such embodiments, the third type of unlabeled nucleotide comprises a mixture of a third type of unlabeled nucleotide comprising a biotin moiety, and a third type of unlabeled nucleotide comprising a DNP moiety. In another embodiment, the second hapten comprises a digoxigenin (DIG) moiety and the second hapten-binding partner comprises an anti-DIG antibody. In some such embodiments, the third type of unlabeled nucleotide comprises a mixture of a third type of unlabeled nucleotide comprising a biotin moiety, and a third type of unlabeled nucleotide comprising a DIG moiety.

### Three-Tag System

In some other embodiments of the kit described herein, the third type of unlabeled nucleotide comprises a third hapten, and the set of affinity reagents further comprises a third affinity reagent comprising a third hapten-binding partner that is capable of specific binding to the third hapten. In a further embodiment, the third hapten is covalently attached to the nucleobase of the third type of unlabeled nucleotide via a cleavable linker. In a further embodiment, the first hapten comprises a biotin moiety and the first hapten-binding partner comprises avidin. In a further embodiment, the avidin is streptavidin. In a further embodiment, the avidin is neutravidin. In a further embodiment, one or more biotin binding sites of the avidin are blocked by a biotin moiety-containing molecule or an analog thereof. In still further embodiments, all remaining biotin binding sites of the avidin are blocked by the biotin moiety-containing molecule or an analog thereof. In some such embodiments, one of the second hapten and the third hapten comprises a DNP moiety, and the other one of the second hapten and the third hapten comprises a DIG moiety. In another embodiment, one of the first hapten and the second hapten comprises a DNP moiety, and the other one of the first hapten and the second hapten comprises a DIG moiety. In some such embodiments, the third hapten comprises a biotin moiety and the third hapten-binding partner comprises avidin. In a further embodiment, the avidin is streptavidin. In a further embodiment, the avidin is neutravidin. In a further embodiment, one or more biotin binding sites of the avidin are blocked by a biotin moiety-containing molecule or an analog thereof. In still further embodiments, all remaining biotin binding sites of the avidin are blocked by the biotin moiety-containing molecule or an analog thereof. In a further embodiment, the third affinity reagent comprises one or more first detectable labels and one or more second detectable labels.

In some embodiments, the detectable label is covalently attached to an affinity reagent by reacting a functional group of the detectable label with a functional group of the hapten-binding partner (e.g., protein tag or antibody). For example, a carboxy group of the detectable label may react with an amino group of the hapten-binding partner (e.g., the amino group of an amino acid moiety of the protein or antibody) to form an amide bond. In one example, one or more fluorescent dyes are covalently attached to one or more lysine moieties on the protein/antibody (e.g., streptavidin). In some embodiments, the average number of detectable labels on the protein/antibody is measured by comparing the ratio of absorption wavelength of the protein/antibody (e.g., absorbance at 280 nm) and the detectable label (e.g., a fluorescent dye). In some embodiments, the average molar ratio of the first detectable label to the second detectable label may be between about 10:1, 9.5:1, 9:1, 8.5:1, 8:1, 7.5:1, 7:1, 6.5:1, 6:1, 5.5:1, 5:1, 4.5:1, 4:1, 3.5:1, 3:1, 2.5:1, 2:1, 1.5:1, 1:1, 1:1.5, 1:2, 1:2.5, 1:3, 1:3.5, 1:4, 1.4.5, 1:5, 1:5.5, 1:6, 1:6.5, 1:7, 1:7.5, 1:8, 1:8.5, 1:9, 1:9.5, and 1:10. In a further embodiment, the third affinity reagent comprises a multi-dye labeled protein assembly system as described herein.

In addition to the biotin-avidin (e.g., streptavidin or neutravidin), DNP/anti-DNP, and DIG/anti-DIG pairing described herein, other hapten and hapten-binding partners may be used the two-tag or three-tag system described herein. Table 1 lists non-limiting examples of haptens and hapten-binding partners.

TABLE 1
Illustrative examples of haptens and hapten-binding partners.

| Hapten | Hapten-binding partner |
|---|---|
| Biotin | Avidin (e.g., streptavidin or neutravidin) |
| Alkyl chloride | HaloTag ® |
| DNP | Anti-DNP antibody |
| DIG | Anti-DIG antibody |
| β-N-acetyl glucosamine (O-GlcNAc) | WGA (lectin) |
| Alkyl guanine | SNAP-Tag ® |
| 3-nitrotyrosine | Anti-nitrotyrosine antibody |
| Nickel or cobalt complex such as Ni- | His-Tag |
| nitrilotriacetic acid (NTA) | |
| Zinc complex | Oligo-aspartate protein |

In any embodiments of the kit described herein, the concentration of each of affinity reagent (e.g., labeled protein tag or labeled antibody) in the affinity reagent mixture may independently range from about 0.01 μM to about 1μM, from about 0.02 μM to about 0.5 μM, or from about 0.05 μM to about 0.25 μM. In further embodiments, the concentration of the affinity reagent may be independently about 0.01 μM, 0.02 μM, 0.03 μM, 0.04 μM, 0.05 μM, 0.06 μM, 0.07 μM, 0.08 μM, μM, 0.11 μM, 0.12 μM, 0.13 μM, 0.14 μM, 0.15 μM, 0.16 μM, 0.17 μM, 0.18 μM, 0.19 μM, μM, 0.22 μM, 0.24 μM, 0.26 μM, 0.28 μM, 0.30 μM, 0.35 μM, 0.40 μM, 0.45 μM, or 0.50 μM, or a range defined by any two of the preceding values.

In any embodiments of the kit described herein, the kit may further comprise a fourth type of unlabeled nucleotide, wherein the fourth type of unlabeled nucleotide is not capable of specific binding to any of the affinity reagents. In a further embodiment, the fourth type of unlabeled nucleotide cannot be excited by either the first light source or the second light source.

In any embodiments of the kit described herein, each of the first type, second type, third type and fourth type of unlabeled nucleotide comprises a 3′ blocking group as described herein. For example, the 3′ blocking group may be an azidomethyl (—CH₂N₃) attached to the 3′ oxygen of the nucleotide. In another example, the 3′ blocking group may be —OCH₂OCH₂CH═CH₂ (AOM) attached to the 3′ carbon of the nucleotide. In another example, 3′ blocking group may be an allyl group (—CH₂CH═CH₂) attached to the 3′ oxygen of the nucleotide.

In any embodiments of the kit described herein, any one or more of the first hapten, the second hapten or the third hapten may be covalently attached to the nucleotide via a cleavable linker described herein. In some embodiments, the cleavable linker may contain one or more azido moieties, one or more allyl moieties or a combination thereof. The linker may further contain spacer such as PEG. In further embodiments, the cleavable linker may be cleavable under the same reaction condition as the 3′ blocking group.

In any embodiments of the kit described herein, the kit may further comprise a DNA polymerase and one or more buffer compositions. In further embodiments, the kit may include one or more unlabeled nucleotides that is not a dark G. For example, the kit may include one or more unlabeled T nucleotide to attenuate the green T signal. Alternatively or additionally, the kit may include one or more labeled nucleotide(s), where the labeled nucleotide(s) may contain a fluorescent label that cannot be excitable by either the first or the second light source.

### Detectable Labels

### A. Protein Assembly System

One aspect of the present disclosure relates to a protein assembly system, comprising:
a first protein labeled with one or more first detectable labels;
one or more hapten-containing linkers covalently attached to the first protein; and
one or more hapten-binding second proteins bound to one or more hapten-containing linkers;
wherein the one or more hapten-binding second proteins are labeled with one or more second detectable labels, and wherein the one or more first detectable labels are spectrally distinguishable from the one or more second detectable labels.

In some embodiments of the protein assembly system described herein, the one or more hapten-binding second proteins bound to the one or more hapten-containing linkers via noncovalent interactions. In other embodiments, the one or more hapten-binding second proteins bound to the one or more hapten-containing linkers via covalent interactions. In some further embodiments, the one or more hapten-containing linkers are biotin moiety-containing linkers, and the one or more hapten-binding second proteins are biotin-binding proteins. In a further embodiment, the one or more hapten-binding second proteins comprise an avidin. In a further embodiment, the avidin is streptavidin. In a further embodiment, the avidin is neutravidin. In a further embodiment, one or more biotin binding sites of the avidin are blocked by a biotin moiety-containing molecule or an analog thereof. In yet a further embodiment, all remaining biotin binding sites of the avidin are blocked by a biotin moiety-containing molecule or an analog thereof.

In some embodiments of the protein assembly system described herein, the first protein comprises an antibody. In further embodiments, the antibody is an anti-DNP antibody or an anti-DIG antibody.

In some embodiments of the protein assembly system described herein, the one or more first detectable labels are excitable by a blue light, and the one or more second detectable labels are excitable by a green light. In some such embodiments, the one or more first detectable labels comprise or are selected from the coumarin dye having the structure of Formula (I) as described herein. In another embodiment, the one or more first detectable labels are excitable by a green light, and the one or more second detectable labels are excitable by a blue light. In further embodiments, the blue light has a wavelength from about 450 nm to about 460 nm. In further embodiments, the green light has a wavelength from about 520 nm to about 540 nm (e.g., about 532 nm).

In some embodiments of the protein assembly system described herein, the first protein is labeled with a plurality of first detectable labels. In further embodiments, the first protein is labeled with at least three, at least four, at least five, at least six, at least seven, at least eight, at least nine, or at least ten first detectable labels.

In some embodiments of the protein assembly system, wherein the system comprises at least two biotin-binding proteins (e.g., three biotin-binding proteins), each biotin-binding protein is labeled with a plurality of second detectable labels. In further embodiments, each biotin-binding protein is labeled with at least three, at least four, at least five, at least six, at least seven, at least eight, at least nine, or at least ten second detectable labels. In further embodiments, one or more of the biotin-binding proteins may comprise an avidin. In a further embodiment, the avidin is streptavidin. In a further embodiment, the avidin is neutravidin.

In some embodiments of the protein assembly system, the biotin moiety-containing linker comprises PEG repeating units (—OCH₂CH₂—)ₙ, where n is an integer from 2 to 30. In some embodiments, n is 5, 6, 7, 8, 9, 10, 11, 12, 13, 14 or 15.

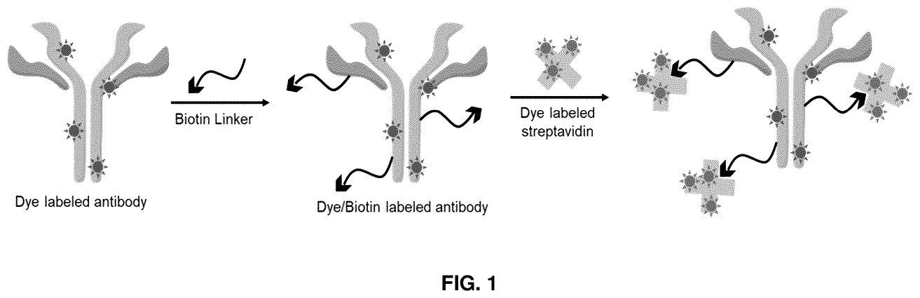

FIG. 1 schematically illustrates a dual protein assembly system according to an embodiment of the present disclosure. The first protein may be an antibody with one or more first detectable labels (e.g., anti-DNP or anti-DIG antibody). The first detectable labels may include, for example, a first dye moiety. The first protein may be labeled with one, two, three, four, five, six, seven, eight, nine, ten, eleven, twelve, thirteen, fourteen, or fifteen detectable labels, though in some embodiments more detectable labels may be included. The one or more hapten-containing linkers may be biotin moiety containing linkers. One or more biotin-containing linkers may be covalently attached to the first protein. For example, one, two, three, four, five, six, seven, eight, nine, or ten biotin moiety containing linkers may be covalently attached to the first protein, though in some embodiments more biotin linkers may be attached to the first protein/antibody. The one or more hapten-binding second proteins may include one or more second detectable labels. For example, the one or more hapten-binding second proteins may comprise or be avidin molecules, for example streptavidin or neutravidin, in accordance with the present embodiment. The second detectable labels may include a second dye moiety. The first dye moiety and second dye moiety may have different excitation and fluorescence emission wavelengths, such that the emitted fluorescence of the first dye moiety is spectrally distinguishable from the emitted fluorescence of the second dye moiety. In some embodiments, the first detectable labels may comprise “blue dyes”, which are excitable by a blue light source having a wavelength between about 450 nm to about 460 nm. In some further embodiments, the second detectable labels may comprise “green dyes”, which are excitable by a green light source having a wavelength between about 520 nm to about 540 nm. The labeled avidin molecules may be introduced and bind to the biotin moiety containing linkers attached to the first protein/antibody. Thus, the protein assembly system may include both a plurality of first detectable labels and a plurality of second detectable labels. In further embodiments, the molar ratio of first protein/antibody to the second protein (e.g., avidin/streptavidin/neutravidin) may be between about 10:1, 9:1, 8:1, 7:1, 6:1, 5:1, 4:1, 3:1, 2:1, 1:1, 1:2, 1:3, 1:4, 1:5, 1:6, 1:7, 1:8, 1:9 and 1:10. In some embodiments, the molar ratio of the first protein/antibody to the second protein is calculated by the molar amount of each component in the reaction to prepare the protein assembly system. In one embodiment, the molar ratio of first protein/antibody (e.g., anti-DNP) to streptavidin is about 1:3. In further embodiments, anti-DNP is labeled, on average, with about or at least about 2, 2.5, 3, 3.5, 4, 4.5, 5, 5.5, 6, 6.5, 7, 7.5, 8, 8.5, 9, 9.5 or 10 blue or green dyes. In further embodiments, each streptavidin is labeled, on average, with about or at least about 2, 2.5, 3, 3.5, 4, 4.5, 5, 5.5, 6, 6.5, 7, 7.5, 8, 8.5, 9, 9.5 or 10 green or blue dyes. In still further embodiments, the remaining biotin binding sites of the streptavidin in the dual protein assembly system are blocked with a biotin moiety containing molecule or an analog thereof such that the dual protein assembly system is only capable of specific binding with a DNP moiety containing nucleotide.

One aspect of the present disclosure relates to a nucleotide conjugate comprising the protein assembly system described herein. In some embodiments, the nucleotide is conjugated with the protein assembly system via noncovalent interaction. In a further embodiment, the protein assembly system is bound to a hapten moiety of the nucleotide, and the hapten moiety of the nucleotide is capable of specific binding to at least one of the proteins of the protein assembly system. In yet a further embodiment, the hapten moiety of the nucleotide comprises a DIG or a DNP moiety. In a further embodiment, the hapten moiety of the nucleotide is covalently attached to the nucleobase via a cleavable linker.

Another aspect of the present disclosure relates to an oligonucleotide or polynucleotide comprising the nucleotide conjugate described herein. In a further embodiment, the oligonucleotide or polynucleotide is at least partially complementary and hybridized to a target polynucleotide immobilized on a surface of a solid support. In a further embodiment, the solid support comprising a plurality of different immobilized target polynucleotides, wherein the density of the immobilized target polynucleotides (i.e., clusters) on the solid support is about or at least about 50,000/mm², 100,000/mm², 150,000/mm², 200,000/mm², 250,000/mm², 300,000/mm², 350,000/mm², or 400,000/mm².

### B. Labeled Avidin

Some aspect of the present disclosure relates to a labeled avidin for use as an affinity reagent described herein, or for use as part of the protein assembly system described herein. In particular, the labeled avidin may be a multi-dye labeled streptavidin or neutravidin, wherein at least one biotin-binding site of the avidin is blocked with a biotin moiety-containing molecule or an analog thereof, and wherein the labeled avidin comprises at least one (or only one) open biotin-binding site (i.e., for binding with the biotin moiety-containing nucleotide, or biotin moiety-containing linker of the protein assembly system).

In some embodiments of the labeled avidin, for example streptavidin or neutravidin, described herein, one to three biotin-binding sites of the avidin are blocked with the biotin moiety-containing molecule or the analog thereof. In some embodiments, one biotin-binding site of the avidin is blocked with the biotin moiety-containing molecule or the analog thereof. In some embodiments, two biotin-binding sites of the avidin are blocked with the biotin moiety-containing molecule or the analog thereof. In some embodiments, three biotin-binding sites of the avidin are blocked with the biotin moiety-containing molecule or the analog thereof. In some embodiments, wherein the biotin moiety-containing molecule or the analog thereof is a free molecule that is not covalently attached to a nucleotide or nucleoside. For example, the biotin moiety-containing molecule or the analog thereof may comprise a free biotin

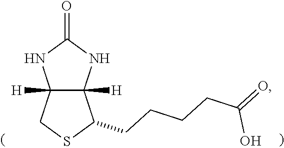 [SMILES: O=C(O)CCCC[C@@H]1SC[C@@H]2NC(=O)N[C@@H]21]

biotin-TEG, dual biotin, PC biotin, desthiobiotin-TEG, biotin azide, or combinations thereof. In other embodiments, the biotin moiety-containing molecule or the analog thereof comprises a biotin moiety that is covalently attached to a nucleotide or nucleoside, optionally via a linker (e.g., a cleavable linker). In some embodiments of the labeled avidin described herein, avidin is labeled with two to eight fluorescent dye moieties. In some embodiments, avidin is labeled with two fluorescent dye moieties. In some embodiments, avidin is labeled with five to seven fluorescent dye moieties.

In some embodiments of the labeled avidin described herein, the fluorescent dye moieties are identical and/or spectrally indistinguishable. In some embodiments, the fluorescent dye moieties of the avidin are excitable by a light source have a wavelength between about 400 nm to about 650 nm. For example, the light source may have a wavelength of about 400 nm, 410 nm, 420 nm, 430 nm, 440 nm, 445 nm, 450 nm, 455 nm, 460 nm, 465 nm, 470 nm, 480 nm, 490 nm, 500 nm, 510 nm, 515 nm, 520 nm, 525 nm, 530 nm, 535 nm, 540 nm, 545 nm, 550 nm, 560 nm, 570 nm, 580 nm, 590 nm, 600 nm, 610 nm, 620 nm, 630 nm, 640 nm or 650 nm, or a range defined by any two of the preceding values. For example, a wavelength in any one of the following ranges: about 440-470 nm, about 450-460 nm, about 510-545 nm, 515-540 nm, 520-535 nm, or 525-535 nm. In some further embodiments, the fluorescent dye moieties are excitable by a light source having a wavelength between about 450 nm to about 460 nm, or between about 520 nm to about 535 nm.

More detail regarding labeled avidin can be found in U.S. application Ser. No. 18/190330, which incorporated herein by reference.

### C. Depolymerizable Labeled Nanoparticles

Another aspect of the present disclosure relates to a labeled nanoparticle, comprising:
a polymer matrix comprising a plurality of detectable labels, wherein the backbone of the polymer comprises one or more cleavable moieties, and wherein the labeled nanoparticle is degradable into smaller polymeric chains upon cleavage of the cleavable moieties.

In some embodiments of the labeled nanoparticle described herein, the plurality of detectable labels comprise or are fluorophores excitable by a light source having a wavelength between a wavelength of about 400 nm to about 650 nm, from about 420 nm to about 600 nm, or from about 450 nm to about 550 nm.

In some embodiments of the labeled nanoparticle described herein, the detectable labels are fluorophores excitable by a light source having a wavelength between a wavelength of about 450 nm to about 460 nm.

In some embodiments of the labeled nanoparticle described herein, the smaller polymeric chains of degraded nanoparticle are water soluble.

In some embodiments of the labeled nanoparticle described herein, the cleavable moieties are chemically degradable, enzymatically degradable, thermally degradable, or photo-degradable.

In some embodiments of the labeled nanoparticle described herein, the cleavable moieties comprise a disulfide, an azido, or an allyl moiety, or combinations thereof.

In some embodiments of the labeled nanoparticle described herein, the polymer comprises a cross-linked or a dendritic polymer, or a liposome.

In some embodiments of the labeled nanoparticle described herein, the polymer comprises polyacrylamide, polyglycolic acid (PGA), polyvinyl alcohol (PVA).

In some embodiments of the labeled nanoparticle described herein, the plurality of detectable labels are covalently attached to the polymer matrix.

In some embodiments of the labeled nanoparticle described herein, the plurality of detectable labels are encapsulated or physically confined within the polymer matrix.

In some embodiments of the labeled nanoparticle described herein, the labeled nanoparticle further comprises one or more proteins attached thereto.

As well density of patterned flow cells used in SBS increases, the size of wells decreases. This leaves less area for template strands. As previous ffNs have been conjugated with one fluorophore per base, the fluorescence signal per well also decreases.

As discussed herein, it is possible to conjugate an ffN with a hapten and flow in a corresponding hapten-binding partner with one or more incorporated fluorophores. In some embodiments, the binding partner is itself conjugated to a nanoparticle or quantum dot to which the one or more incorporated fluorophores are conjugated. However, these nanoparticles may remain adsorbed on DNA clusters of the flow cell even after wash steps, creating high background fluorescence. A nanoparticle that can be depolymerized may be more easily washed from the DNA clusters.

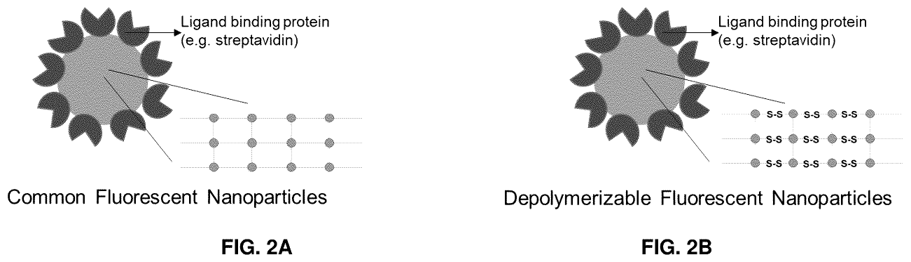

FIG. 2A schematically illustrates a non-depolymerizable nanoparticle while FIG. 2B schematically illustrates an example depolymerizable fluorescent nanoparticle in accordance with the present embodiment. Each nanoparticle may include a polymer matrix. Additionally, the nanoparticles may include a ligand binding protein (i.e., a hapten-binding partner) via which the nanoparticle may bind to one or more dye-labeled molecules. In alternative embodiments, the dye-labeling may be attached to the nanoparticle covalently. Unlike the common nanoparticle, the polymer matrix of the depolymerizable fluorescent nanoparticles may include cleavable moieties. In the example illustrated in FIG. 2B, the cleavable moiety may be a disulfide bond. If, during SBS, a suitable cleave mix is introduced to the depolymerizable fluorescent nanoparticles, the nanoparticles may disassemble into small soluble chains which may be washed away.

Though FIG. 2B depicts the nanoparticle including disulfide bond, other cleavable bonds may be used in a depolymerizable nanoparticle. In some embodiments, disulfide bonds may be cleaved by a reducing agent. In some embodiments, allyl bonds may be cleaved by a Pd(0) complex (e.g., Tris(3,3′,3″-phosphinidynetris(benzenesulfonato)palladium(0) nonasodium salt nonahydrate), or the same palladium cleavage reagents described herein for cleaving the 3′ blocking group/cleavable linker. In some embodiments, azidomethyl bonds may be cleaved by tris(2-carboxyethyl)phosphine (TCEP) or tris(hydroxypropyl)phosphine (THP or THPP). In some embodiments, diol bonds can be cleaved by NaIO₄. In some embodiments, polyglycolic acid bonds can be cleaved by H₂O or by enzymes with esterase activity. In some embodiments, polyvinyl alcohol (PVA) bonds can be cleaved by heat, enzymatic action, or oxidation. In some embodiments, ester bonds can be cleaved by hydrolysis, heat, or alkaline conditions. In some embodiments, photocleavable bonds can be cleaved by exposure to UV light. In some embodiments, the nanoparticle may include a polymersome. The polymersome can be degradable at particular pH ranges, by surfactants, and/or by hydrolysis. In some embodiments, the nanoparticle may include a liposome. The liposome can be degradable by at particular pH ranges, by surfactants, at particular temperature ranges, and/or by hydrolysis. Other suitable cleavable bonds may be used.

### Fluorescent Dyes

Various fluorescent dyes may be used in the present disclosure as detectable labels for the affinity reagents described herein, in particularly those dyes that may be excitation by a blue light or a green light. These dyes may also be referred to as “blue dyes” and “green dyes” respectively. Examples of various type of blue dyes, including but not limited to coumarin dyes, chromenoquinoline dyes, naphthalimide dyes, and bisboron containing heterocycles are disclosed in U.S. Publication Nos. 2018/0094140, 2018/0201981, 2020/0277529, 2020/0277670, 2021/0188832 and 2022/0033900, and U.S. Ser. Nos. 17/550271, 17/736688, 18/190531, 63/356412 and 63/492896, each of which is incorporated by reference in its entirety. Non-limiting examples of the blue dyes include:

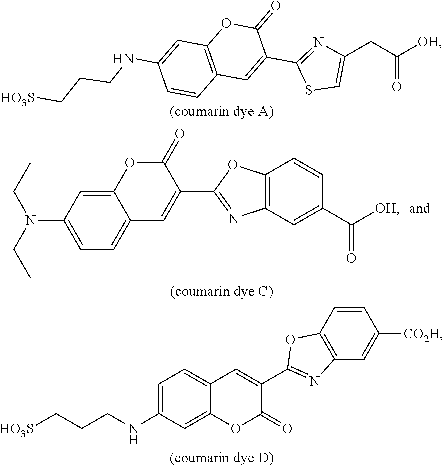

and salts, mesomeric forms, and optionally substituted analogs thereof. For example, analogs with —SO₃H substitution on the alkyl group(s).

Examples of green dyes including cyanine or polymethine dyes disclosed in International Publication Nos. WO2013/041117, WO2014/135221, WO2016/189287, WO2017/051201 and WO2018/060482A1, each of which is incorporated by reference in its entirety. Non-limiting examples of the green dyes include:

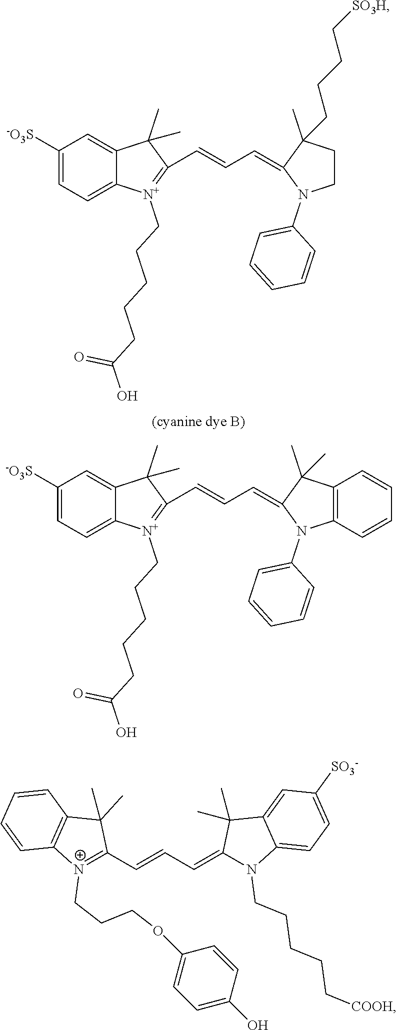

and salts, mesomeric forms, and optionally substituted analogs thereof.

In some embodiments, the fluorescent dyes described herein may be further modified to introduce one or more substituents (such as —SO₃H, —OH, —C(O)OH, —C(O)OR, where R is unsubstituted or substituted C₁-C₆ alkyl) to improve the hydrophilicity of the dyes when the dyes are conjugated with the antibody/protein (i.e., hapten-binding partner) described herein, while maintaining the signal intensity of the dye. In some such embodiments, the first detectable label described in the kits and protein assembly system of the present disclosure may be a coumarin dye having the structure of Formula (I):

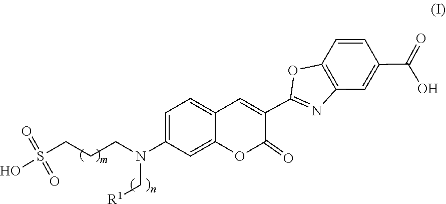

wherein m is an integer of 1, 2 or 3;
n is an integer of 1, 2, 3, 4 or 5;
R¹ is —C(O)OC₁-C₆ alkyl, —SO₃H, —OH, optionally substituted 5 to 10 membered heteroaryl, optionally substituted 3 to 10 membered heterocyclyl, optionally substituted phenyl, or —OR^{x}, wherein Rₓ is optionally substituted 5 to 10 membered heteroaryl, optionally substituted 3 to 10 membered heterocyclyl, or optionally substituted phenyl. In some embodiments, m is 1. In some embodiments, n is 1. In some other embodiments, n is 3. In further embodiments, m is 1 and n is 3. In one embodiment, R¹ is —C(O)OₜBu. In another embodiment, R¹ is —SO₃H. In another embodiment, R¹ is furyl (e.g., 2-furyl). In another embodiment, R¹ is

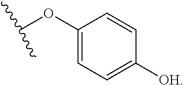 [SMILES: *Oc1ccc(O)cc1]

Further non-limiting embodiments of the coumarin dyes include:

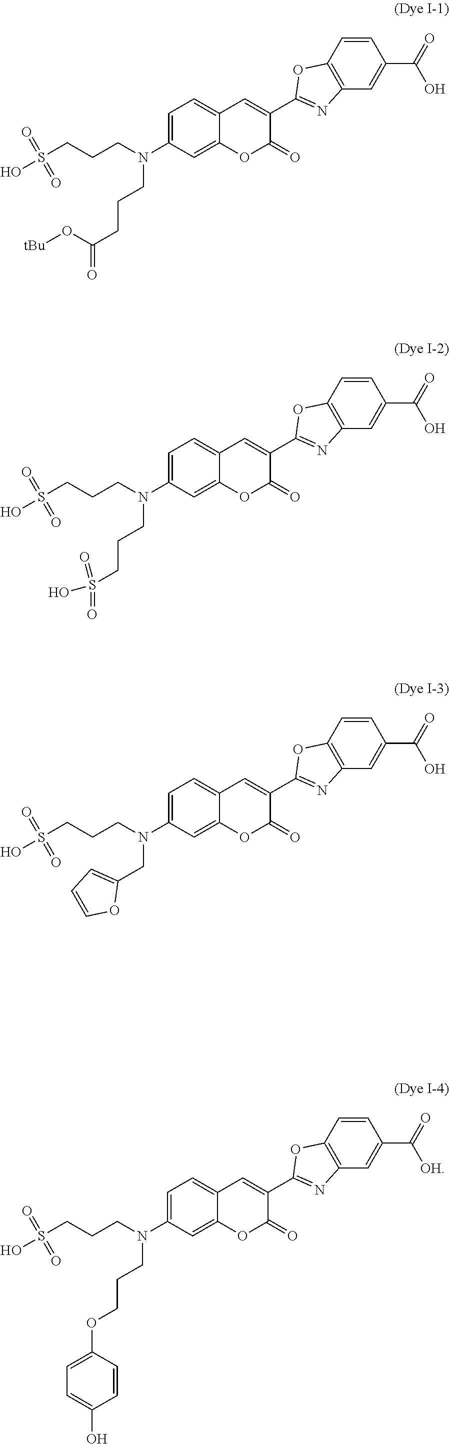 [SMILES: CC(C)(C)OC(=O)CCCN(CCCS(=O)(=O)O)c1ccc2cc(-c3nc4cc(C(=O)O)ccc4o3)c(=O)oc2c1 ; O=C(O)c1ccc2oc(-c3cc4ccc(N(CCCS(=O)(=O)O)CCCS(=O)(=O)O)cc4oc3=O)nc2c1 ; O=C(O)c1ccc2oc(-c3cc4ccc(N(CCCS(=O)(=O)O)Cc5ccco5)cc4oc3=O)nc2c1 ; O=C(O)c1ccc2oc(-c3cc4ccc(N(CCCOc5ccc(O)cc5)CCCS(=O)(=O)O)cc4oc3=O)nc2c1]

When the dye forms a covalent bond with the affinity reagent (e.g., avidin, protein or antibody), the carboxy group of the dye may react with an amino functional group of the affinity reagent to form an amide bond. For example, the dye of Formula (I) comprises the moiety

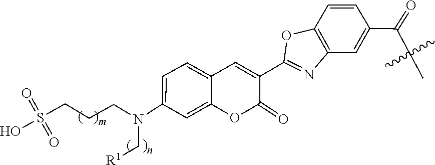

after being covalently bound to the affinity reagent.

### 3′ Blocking Groups

The nucleotide described herein may also have a 3′ blocking group covalently attached to the deoxyribose sugar of the nucleotide. Various 3′ blocking group are disclosed in WO2002/029003, WO2004/018497 and WO2014/139596. For example, the blocking group may be azidomethyl (—CH₂N₃) or substituted azidomethyl (e.g., —CH(CHF₂)N₃ or CH(CH₂F)N₃), or allyl, each connecting to the 3′-oxygen atom of the deoxyribose moiety. In some embodiments, the 3′ blocking group is azidomethyl, forming 3′-OCH₂N₃ with the 3′ carbon of the ribose or deoxyribose.

Additional 3′ blocking groups are disclosed in U.S. Publication No. 2020/0216891 A1, which is incorporated by reference in its entirety. Non-limiting examples of the acetal blocking group

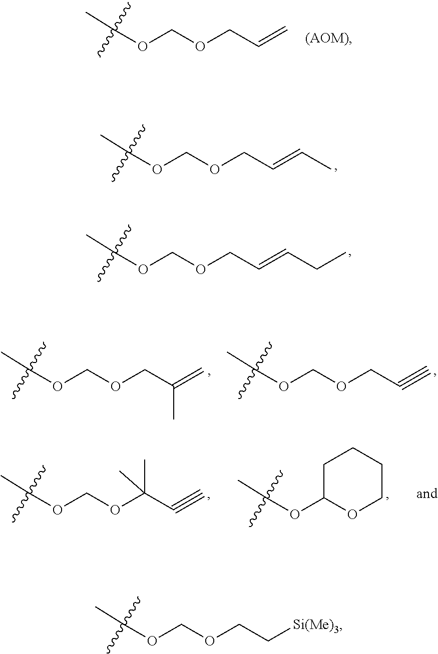

each covalently attached to the 3′ carbon of the deoxyribose.

### Deprotection of the 3′ Blocking Groups

In some embodiments, the 3′ hydroxy protecting group such as azidomethyl may be removed or deprotected by using a water-soluble phosphine reagent to generate a free 3′-OH. Non-limiting examples include tris(hydroxymethyl)phosphine (THMP), tris(hydroxyethyl)phosphine (THEP) or tris(hydroxypropyl)phosphine (THP or THPP). 3′-acetal blocking groups described herein may be removed or cleaved under various chemical conditions. For 3′ blocking groups that contain an allyl moiety, non-limiting cleaving condition includes a Pd(II) complex, such as Pd(OAc)₂ or allylPd(II) chloride dimer, in the presence of a phosphine ligand, for example tris(hydroxymethyl)phosphine (THMP), or tris(hydroxypropyl)phosphine (THP or THPP). For those blocking groups containing an alkynyl group (e.g., an ethynyl), they may also be removed by a Pd(II) complex (e.g., Pd(OAc)₂ or allyl Pd(II) chloride dimer) in the presence of a phosphine ligand (e.g., THP or THMP).

### Palladium Cleavage Reagents

In some other embodiments, the 3′ blocking group such as allyl or AOM as described herein may be cleaved by a palladium catalyst. In some such embodiments, the Pd catalyst is water soluble. In some such embodiments, is a Pd(0) complex (e.g., Tris(3,3′,3″-phosphinidynetris(benzenesulfonato)palladium(0) nonasodium salt nonahydrate). In some instances, the Pd(0) complex may be generated in situ from reduction of a Pd(II) complex by reagents such as alkenes, alcohols, amines, phosphines, or metal hydrides. Suitable palladium sources include Na₂PdC₁₄, Li₂PdC₁₄, Pd(CH₃CN)₂C₁₂, (PdCl(C₃H₅))₂, [Pd(C₃H₅)(THP)]Cl, [Pd(C₃H₅)(THP)₂]Cl, Pd(OAc)₂, Pd(Ph₃)₄, Pd(dba)₂, Pd(Acac)₂, PdCl₂(COD), Pd(TFA)₂, Na₂PdBr₄, K₂PdBr₄, PdCl₂, PdBr₂, and Pd(NO₃)₂. In one such embodiment, the Pd(0) complex is generated in situ from Na₂PdCl₄ or K₂PdCl₄. In another embodiment, the palladium source is allyl palladium(II) chloride dimer [(PdCl(C₃H₅))₂]. In some embodiments, the Pd(0) complex is generated in an aqueous solution by mixing a Pd(II) complex with a phosphine. Suitable phosphines include water soluble phosphines, such as tris(hydroxypropyl)phosphine (THP), tris(hydroxymethyl)phosphine (THMP), 1,3,5-triaza-7-phosphaadamantane (PTA), bis(p-sulfonatophenyl)phenylphosphine dihydrate potassium salt, tris(carboxyethyl)phosphine (TCEP), and triphenylphosphine-3,3′,3″-trisulfonic acid trisodium salt.

In some embodiments, the palladium catalyst is prepared by mixing [(Allyl)PdCl]₂ with THP in situ. The molar ratio of [(Allyl)PdCl]₂ and the THP may be about 1:1, 1:1.5, 1:2, 1:2.5, 1:3, 1:3.5, 1:4, 1:4.5, 1:5, 1:5.5, 1:6, 1:6.5, 1:7, 1:7.5, 1:8, 1:8.5, 1:9, 1:9.5 or 1:10. In one embodiment, the molar ratio of [(Allyl)PdCl]₂ to THP is 1:10. In some other embodiment, the palladium catalyst is prepared by mixing a water soluble Pd reagent such as Na₂PdCl₄ or K₂PdCl₄ with THP in situ. The molar ratio of Na₂PdCl₄ or K₂PdCl₄ and THP may be about 1:1, 1:1.5, 1:2, 1:2.5, 1:3, 1:3.5, 1:4, 1:4.5, 1:5, 1:5.5, 1:6, 1:6.5, 1:7, 1:7.5, 1:8, 1:8.5, 1:9, 1:9.5 or 1:10. In one embodiment, the molar ratio of Na₂PdCl₄ or K₂PdCl₄ to THP is about 1:3. In another embodiment, the molar ratio of Na₂PdCl₄ or K₂PdCl₄ to THP is about 1:3.5. In yet another embodiment, the molar ratio of Na₂PdCl₄ or K₂PdCl₄ to THP is about 1:2.5. In some further embodiments, one or more reducing agents may be added, such as ascorbic acid or a salt thereof (e.g., sodium ascorbate). In some embodiments, the cleavage mixture may contain additional buffer reagents, such as a primary amine, a secondary amine, a tertiary amine, a carbonate salt, a phosphate salt, or a borate salt, or combinations thereof. In some further embodiments, the buffer reagent comprises ethanolamine (EA), tris(hydroxymethyl)aminomethane (Tris), glycine, sodium carbonate, sodium phosphate, sodium borate, 2-dimethylethanolamine (DMEA), 2-diethylethanolamine (DEEA), N,N,N′,N′-tetramethylethylenediamine(TEMED), or N,N,N′,N′-tetraethylethylenediamine (TEEDA), or 2-piperidine ethanol (also known as (2-hydroxyethyl)piperidine, having the structure

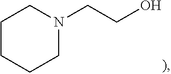

or combinations thereof. In one embodiment, the buffer reagent comprises or is DEEA. In another embodiment, the buffer reagent comprises or is (2-hydroxyethyl)piperidine. In another embodiment, the buffer reagent contains one or more inorganic salts such as a carbonate salt, a phosphate salt, or a borate salt, or combinations thereof. In one embodiment, the inorganic salt is a sodium salt.

### Cleavable Linkers

In some embodiments, the hapten moiety of nucleotide described herein is covalently attached to the nucleobase of the nucleotide via a cleavable linker. Use of the term “cleavable linker” is not meant to imply that the whole linker is required to be removed. The cleavage site can be located at a position on the linker that ensures that part of the linker remains attached to the dye and/or substrate moiety after cleavage. Cleavable linkers may be, by way of non-limiting example, electrophilically cleavable linkers, nucleophilically cleavable linkers, photocleavable linkers, cleavable under reductive conditions (for example disulfide or azide containing linkers), oxidative conditions, cleavable via use of safety-catch linkers and cleavable by elimination mechanisms. The use of a cleavable linker to attach the dye compound to a substrate moiety ensures that the label can, if required, be removed after detection, avoiding any interfering signal in downstream steps.

Useful linker groups may be found in PCT Publication No. WO2004/018493 (herein incorporated by reference), examples of which include linkers that may be cleaved using water-soluble phosphines or water-soluble transition metal catalysts formed from a transition metal and at least partially water-soluble ligands. In aqueous solution the latter form at least partially water-soluble transition metal complexes. Such cleavable linkers can be used to connect bases of nucleotides to labels such as the dyes set forth herein.

Particular linkers include those disclosed in PCT Publication No. WO2004/018493 (herein incorporated by reference) such as those that include moieties of the formulae:

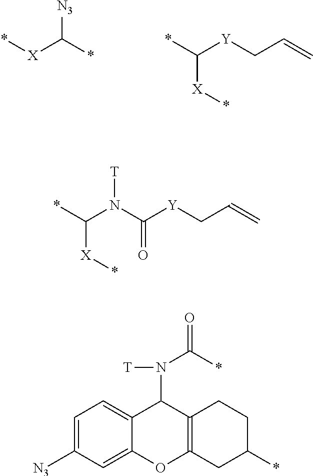

(wherein X is selected from the group comprising O, S, NH and NQ wherein Q is a C₁₋₁₀ substituted or unsubstituted alkyl group, Y is selected from the group comprising O, S, NH and N(allyl), T is hydrogen or a C₁-C₁₀ substituted or unsubstituted alkyl group and * indicates where the moiety is connected to the remainder of the nucleotide or nucleoside). In some aspect, the linkers connect the bases of nucleotides to labels such as, for example, the dye compounds described herein.

Additional examples of linkers include those disclosed in U.S. Publication No. 2016/0040225 (herein incorporated by reference), such as those include moieties of the formulae:

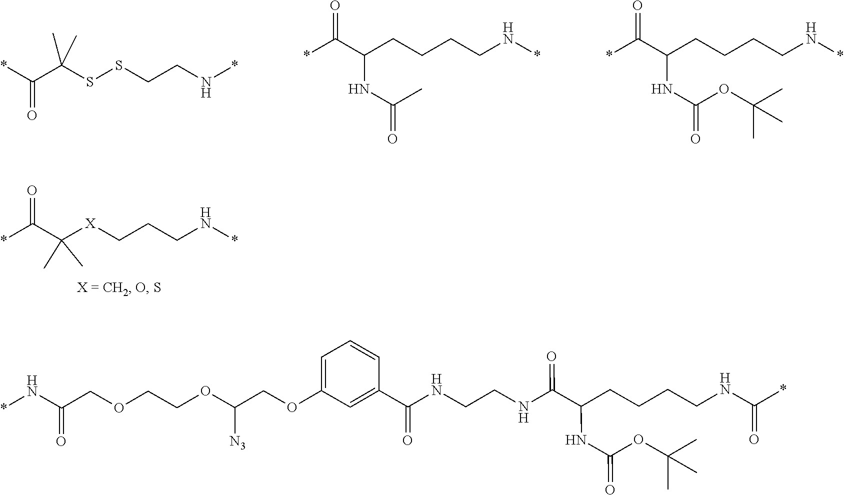

(wherein * indicates where the moiety is connected to the remainder of the nucleotide or nucleoside). The linker moieties illustrated herein may comprise the whole or partial linker structure between the nucleotides/nucleosides and the labels.

Additional examples of linkers are disclosed in U.S. Publication No. 2020/0216891 A1, which is incorporated by reference in its entirety:

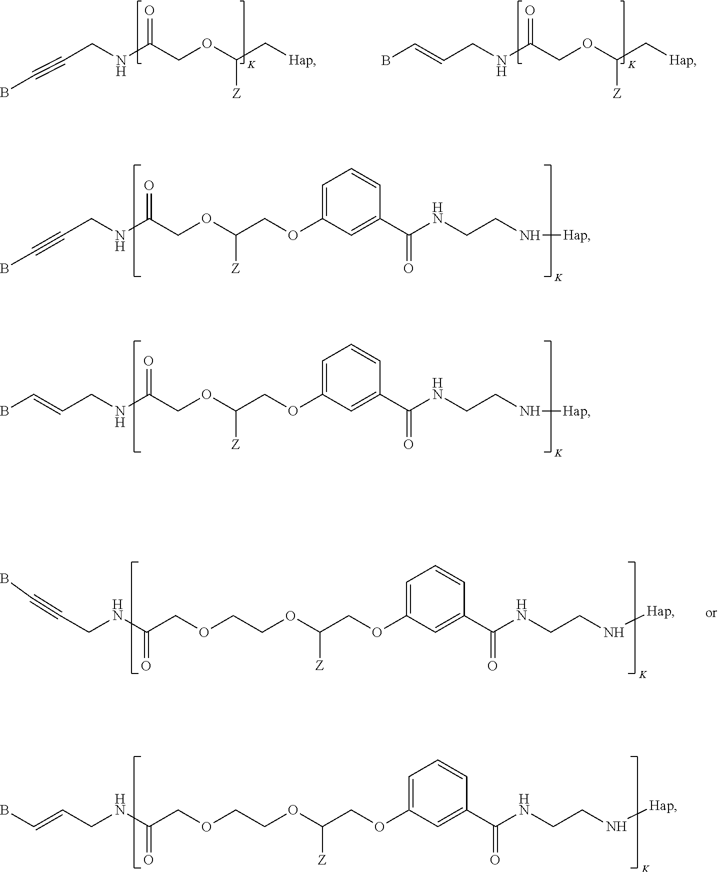

wherein B is a nucleobase; n is 1, 2, 3, 4, 5; k is 1; Z is —N₃ (azido), —O-C₁-C₆ alkyl, —O-C₂-C₆ alkenyl, or —O-C₂-C₆ alkynyl; and Hap comprises a hapten moiety described herein, which may contain additional linker structure. One of ordinary skill in the art understands that the hapten moiety described herein is covalently bound to the linker by reacting a functional group of the hapten compound (e.g., carboxyl) with a functional group of the linker (e.g., amino). In one embodiment, the cleavable linker comprises

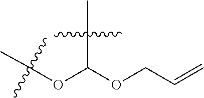 [SMILES: *OC(*)OCC=C]

(“AOL” linker moiety) where Z is —O-allyl. For the purpose of the present disclosure, the nucleotide may contain multiple cleavable linkers repeating units (e.g., k is 1, 2, 3, 4 5, 6, 7, 8, 9 or 10).

An hapten moiety may be attached to any position on the nucleotide base, for example, through a linker. In particular embodiments, Watson-Crick base pairing can still be carried out for the resulting analog. Particular nucleobase labeling sites include the C₅ position of a pyrimidine base or the C₇ position of a 7-deaza purine base. As described above a linker group may be used to covalently attach a dye to the nucleotide.

In particular embodiments, the unlabeled nucleotide may be enzymatically incorporable and enzymatically extendable. Accordingly, a linker moiety may be of sufficient length to connect the nucleotide to the compound such that the compound does not significantly interfere with the overall binding and recognition of the nucleotide by a nucleic acid replication enzyme. Thus, the linker can also comprise a spacer unit, such as one or more PEG unit(s) (—OCH₂CH₂—)ₙ, where n is an integer of 1-20, for example 1, 2, 3, 4, 5, 6, 7, 8, 9, 10, 11, 12, 13, 14 or 15. The spacer distances, for example, the nucleotide base from a cleavage site or label.

A hapten containing unlabeled nucleosides or nucleotides described herein may have the formula:

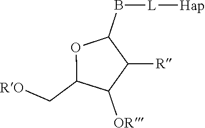

where Hap is a hapten moiety described herein; B is a nucleobase, such as, for example uracil, thymine, cytosine, adenine, 7-deaza adenine, guanine, 7-deaza guanine, and the like; L is an optional linker which may or may not be present; R′ can be H, or -OR' is monophosphate, diphosphate, triphosphate, thiophosphate, a phosphate ester analog, —O— attached to a reactive phosphorous containing group, or —O— protected by a blocking group; R″ is H or OH; and R′″ is H, a 3′ hydroxy blocking group described herein, or —OR′ forms a phosphoramidite. Where —OR′″ is phosphoramidite, R′ is an acid-cleavable hydroxy protecting group which allows subsequent monomer

coupling under automated synthesis conditions. In some further embodiments, B comprises

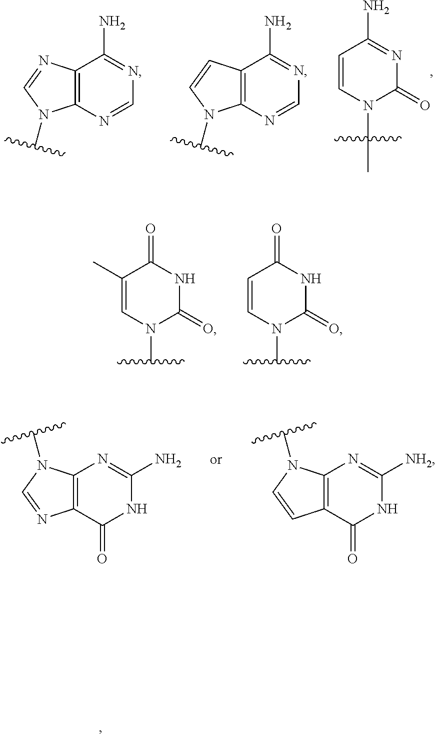

or optionally substituted derivatives and analogs thereof. In some further embodiments, the nucleobase comprises the structure

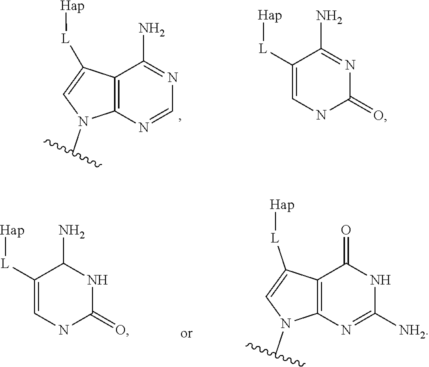

In yet another alternative embodiment, there is no blocking group on the 3′ carbon of the pentose sugar and the labeled avidin attached to the base via a linker, for example, can be of a size or structure sufficient to act as a block to the incorporation of a further nucleotide. Thus, the block can be due to steric hindrance or can be due to a combination of size, charge and structure, whether or not the dye is attached to the 3′ position of the sugar.

The use of a blocking group allows polymerization to be controlled, such as by stopping extension when an unlabeled nucleotide is incorporated. If the blocking effect is reversible, for example, by way of non-limiting example by changing chemical conditions or by removal of a chemical block, extension can be stopped at certain points and then allowed to continue.

In a particular embodiment, the linker (between the hapten moiety and nucleotide) and blocking group are both present and are separate moieties. In particular embodiments, the linker and blocking group are both cleavable under the same or substantially similar conditions. Thus, deprotection and deblocking processes may be more efficient because only a single treatment will be required to remove both the dye compound and the blocking group. However, in some embodiments a linker and blocking group need not be cleavable under similar conditions, instead being individually cleavable under distinct conditions.

Non-limiting exemplary unlabeled nucleotides as described herein include:

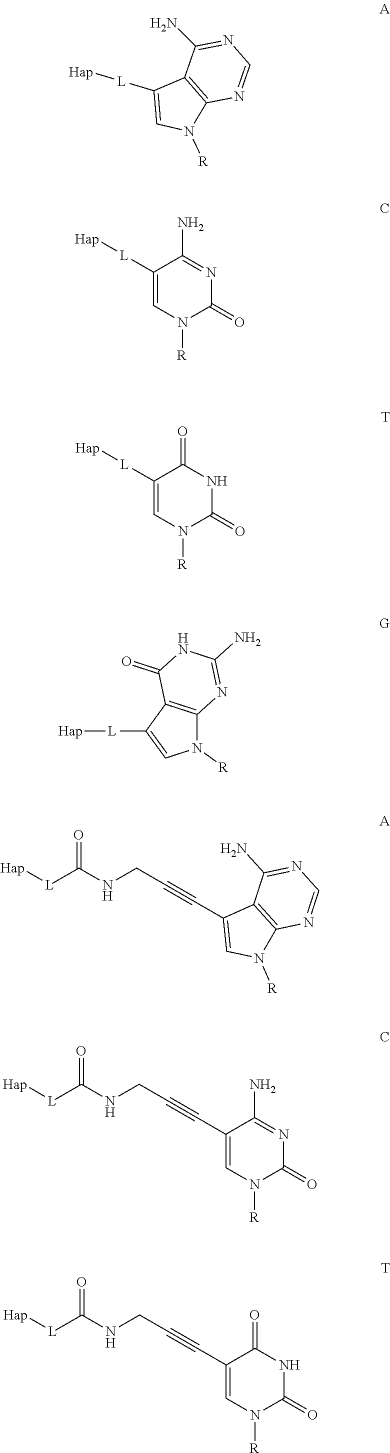

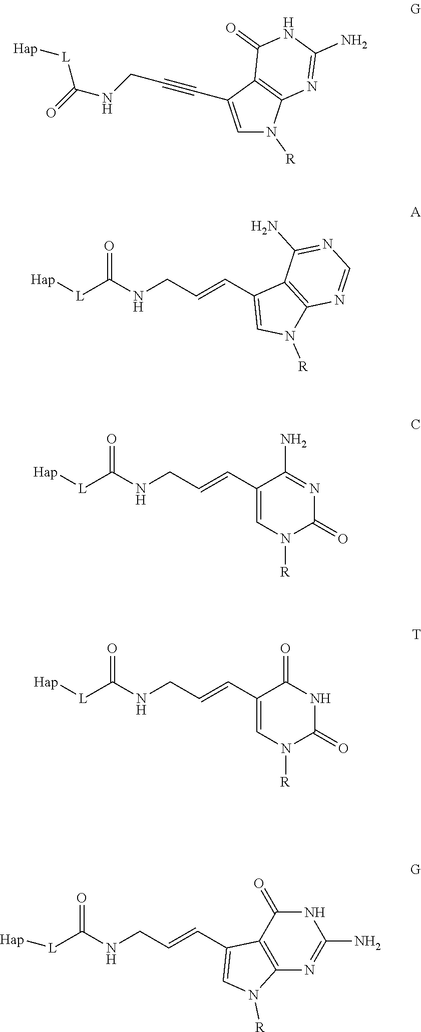

wherein L represents a linker, including a cleavable linker described herein; R represents a ribose or deoxyribose moiety as described above, or a ribose or deoxyribose moiety with the 5′ position substituted with mono-, di- or tri- phosphates; Hap represents a hapten moiety described herein.

In some embodiments, non-limiting exemplary unlabeled nucleotide containing a hapten moiety covalently attached via a cleavable linker are shown below:

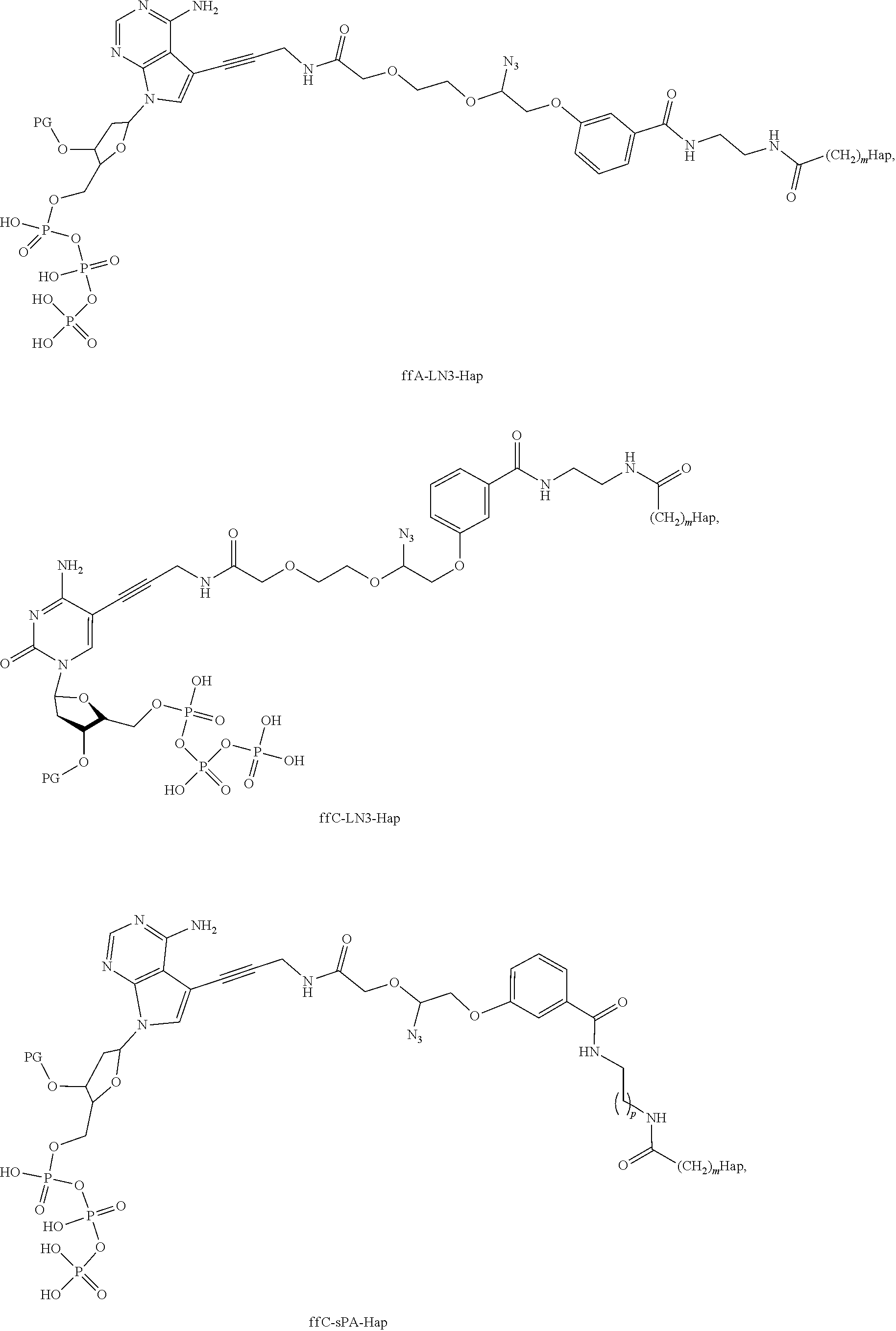

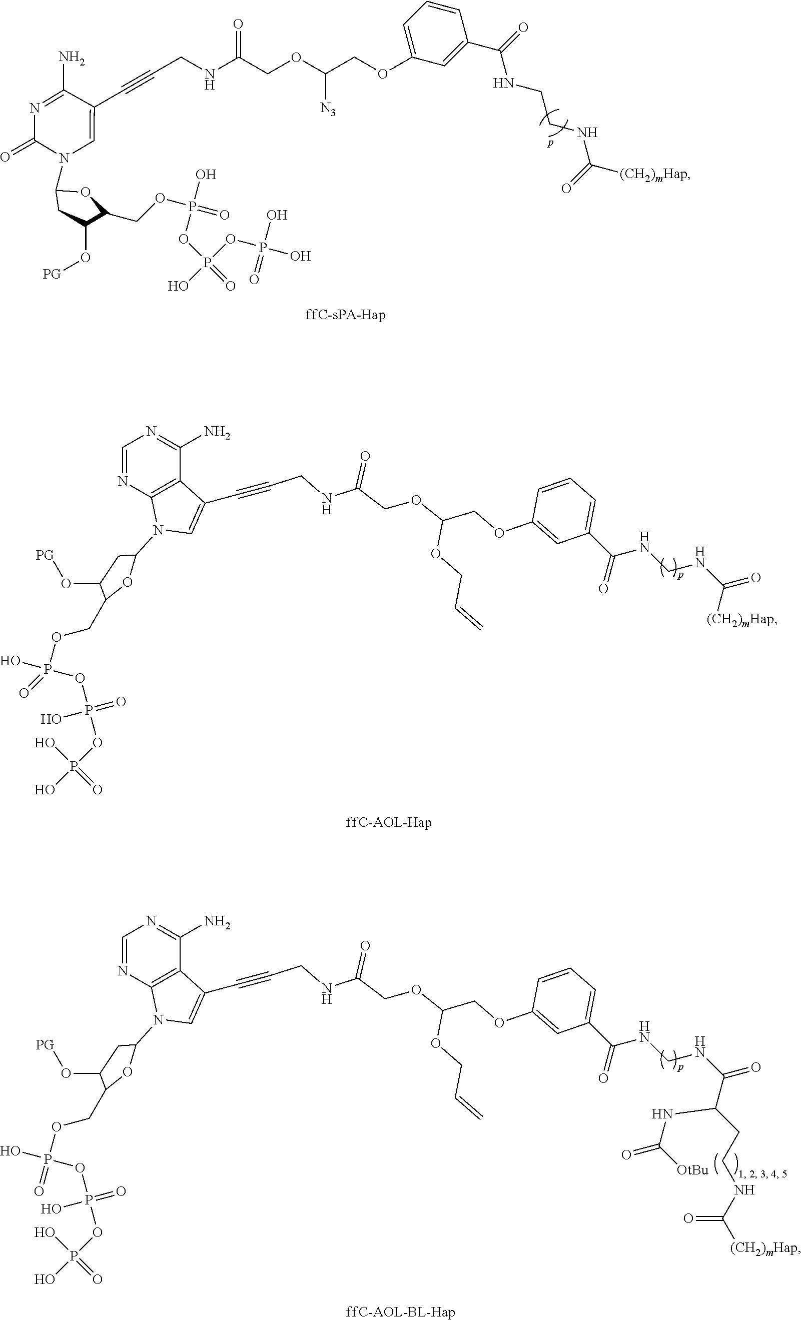

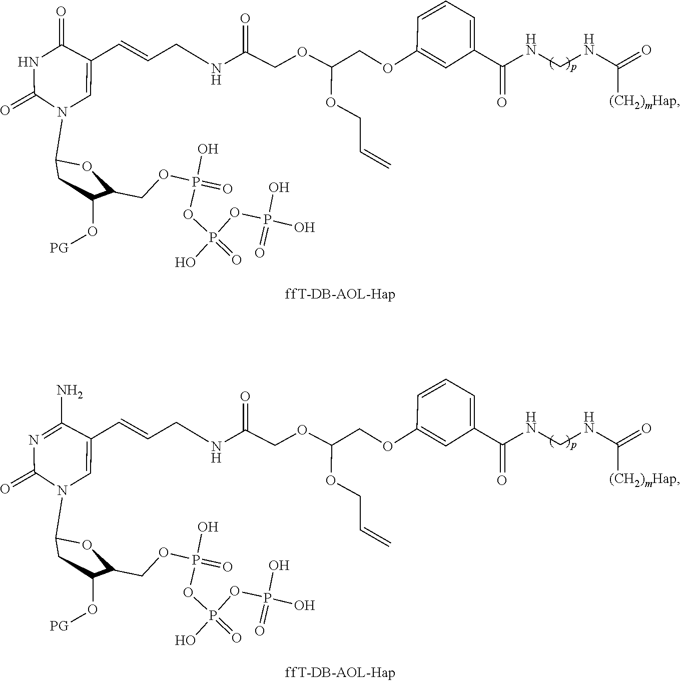

wherein PG stands for the 3′ blocking groups described herein; p is an integer of 1, 2, 3, 4, 5, 6, 7, 8, 9, or 10; and m is 0, 1, 2, 3, 4, or 5. In one embodiment, —O—PG is AOM. In another embodiment, —O—PG is —O-azidomethyl. In one embodiment, k is 5. In some further embodiments, p is 1, 2 or 3; and m is 5.

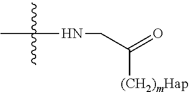

refers to the connection point of the hapten moiety with the cleavable linker as a result of a reaction between an amino group of the linker moiety and the carboxyl group of the hapten. In further embodiments, the nucleotide may be attached to the hapten moiety via more than one of the same cleavable linkers (such as LN3-LN3, sPA-sPA, AOL-AOL). In other embodiments, the nucleotide may be attached to the hapten moiety via two or more different cleavable linkers (such sPA-LN3, sPA-sPA-LN3, sPA-LN3-LN3, etc.). In any embodiments of the labeled nucleotide described herein, the nucleotide is a nucleotide triphosphate. In further embodiments, the nucleotide has a 2′ deoxyribose.

### Methods of Sequencing

Additional aspect of the present disclosure relates to a method for determining the sequences of a plurality of different target polynucleotides in parallel, comprising:
(a) contacting a solid support with a solution comprising sequencing primers under hybridization conditions, wherein the solid support comprises a plurality of different target polynucleotides immobilized thereon; and the sequencing primers are complementary to at least a portion of the target polynucleotides;
(b) contacting the solid support with an aqueous solution comprising DNA polymerase and one or more of four different types of unlabeled nucleotides (e.g., an incorporation mixture containing A, C, G, T or U) under conditions suitable for DNA polymerase-mediated primer extension, and incorporating one type of nucleotides into the sequencing primers to produce extended copy polynucleotides; wherein
   each of the four types of nucleotides comprises a 3′ blocking group;
   the first type of unlabeled nucleotide comprises a first hapten;
   the second type of unlabeled nucleotide comprises a second hapten;
(c) contacting the extended copy polynucleotides with a set of affinity reagents under conditions wherein one affinity reagent binds specifically to the incorporated unlabeled nucleotides to provide labeled extended copy polynucleotides;
(d) imaging the solid support and performing one or more fluorescent measurements of the extended copy polynucleotides; and
(e) removing the 3′ blocking group of the incorporated nucleotides;
wherein the set of affinity reagents comprises:
   a first affinity reagent comprising a first hapten-binding partner that is capable of specific binding to the first type of unlabeled nucleotides; and
   a second affinity reagent comprising a second hapten-binding partner that is capable of specific binding to the second type of unlabeled nucleotides;
   wherein the first affinity reagent comprises one or more first detectable labels that are excitable by a first excitation light source, the second affinity reagent comprises one or more second detectable labels that are excitable by a second excitation light source, and wherein the one or more first detectable labels are spectrally distinguishable from the one or more second detectable labels; and
wherein one of the first excitation light source and the second excitation light source has a wavelength of about 450 nm to about 460 nm, and the other one of the first excitation light source and the second excitation light source has a wavelength of about 520 nm to about 540 nm.

In some embodiments of the sequencing method described herein, the first hapten is covalently attached to the nucleobase of the first type of unlabeled nucleotide via a cleavable linker. In some further embodiments, the second hapten is covalently attached to the nucleobase of the second type of unlabeled nucleotide via a cleavable linker.

### Two-Tag Sequencing

In some embodiments of the sequencing method described herein, the third type of unlabeled nucleotide comprises a mixture of the third type of unlabeled nucleotide comprising the first hapten and the third type of unlabeled nucleotide comprising the second hapten, and wherein both the first affinity reagent and the second affinity reagent are capable of specific binding to the third type of unlabeled nucleotide. In some further embodiments, each of the first hapten and the second hapten is covalently attached to the nucleobase of the third type of nucleotide via a cleavable linker. In this instance, the incorporation of the first type of nucleotide is determined by a signal state in the first fluorescent measurement and a dark state in the second fluorescent measurement. The incorporation of the second type of nucleotide is determined by a signal state in the first fluorescent measurement and a dark state in the second fluorescent measurement. The incorporation of the third type of nucleotide is determined by a signal state in the first fluorescent measurement and a signal state in the second fluorescent measurement. The incorporation of the fourth type of nucleotide is determined by a dark state in the first fluorescent measurement and a dark state in the second fluorescent measurement. The sequencing system described herein may also be referred to as two-tag system as there are two affinity reagents labeled with spectrally distinguishable detectable labels respectively.

In some embodiments of the two-tag sequencing method described herein, the first hapten comprises a biotin moiety and the first hapten-binding partner comprises an avidin. In a further embodiment, the avidin is streptavidin. In a further embodiment, the avidin is neutravidin. In further embodiments, one or more biotin binding sites of the avidin are blocked by a biotin moiety-containing molecule or an analog thereof. In some embodiments, the second hapten comprises a DNP moiety and the second hapten-binding partner comprises an anti-DNP antibody. In some embodiments, the third type of unlabeled nucleotide comprises a mixture of a third type of unlabeled nucleotide comprising a biotin moiety, and a third type of unlabeled nucleotide comprising a DNP moiety. In other embodiments, the second hapten comprises a DIG moiety and the second hapten-binding partner comprises an anti-DIG antibody. In other embodiments, the third type of unlabeled nucleotide comprises a mixture of a third type of unlabeled nucleotide comprising a biotin moiety, and a third type of unlabeled nucleotide comprising a DIG moiety.

### Three-Tag Sequencing

In some other embodiments of the method described herein, the third type of unlabeled nucleotide comprises a third hapten, and the set of affinity reagents further comprises a third affinity reagent comprising a third hapten-binding partner that is capable of specific binding to the third hapten. In some such embodiments, the third hapten is covalently attached to the nucleobase of the third type of unlabeled nucleotide via a cleavable linker. In this instance, the incorporation of the first type of nucleotide is determined by a signal state in the first fluorescent measurement and a dark state in the second fluorescent measurement. The incorporation of the second type of nucleotide is determined by a signal state in the first fluorescent measurement and a dark state in the second fluorescent measurement. The incorporation of the third type of nucleotide is determined by a signal state in the first fluorescent measurement and a signal state in the second fluorescent measurement. The incorporation of the fourth type of nucleotide is determined by a dark state in the first fluorescent measurement and a dark state in the second fluorescent measurement. The sequencing system described herein may also be referred to as three-tag system as there are three affinity reagents labeled with spectrally distinguishable detectable labels respectively.

In some embodiments of the three-tag sequencing method described herein, the first hapten comprises a biotin moiety and the first hapten-binding partner comprises avidin. In a further embodiment, the avidin is streptavidin. In a further embodiment, the avidin is neutravidin. In further embodiments, one or more biotin binding sites of the avidin are blocked by a biotin moiety-containing molecule or an analog thereof. In some embodiments, one of the second hapten and the third hapten comprises a DNP moiety, and the other one of the second hapten and the third hapten comprises a DIG moiety. For example, the second hapten comprises a DNP moiety and the second hapten-binding partner comprises an anti-DNP antibody. The third hapten comprises a DIG moiety and the third hapten-binding partner comprises an anti-DIG antibody.

In some other embodiments of the three-tag sequencing method described herein, one of the first hapten and the second hapten comprises a DNP moiety, and the other one of the first hapten and the second hapten comprises a DIG moiety. For example, the first hapten comprises a DNP moiety and the first hapten-binding partner comprises an anti-DNP antibody. The second hapten comprises a DIG moiety and the second hapten-binding partner comprises an anti-DIG antibody. In some further embodiments, the third hapten comprises a biotin moiety and the third hapten-binding partner comprises avidin. In a further embodiment, the avidin is streptavidin. In a further embodiment, the avidin is neutravidin. In some further embodiments, one or more biotin binding sites of the avidin are blocked by a biotin moiety-containing molecule or an analog thereof.

In some embodiments of the three-tag sequencing method described herein, the third affinity reagent comprises one or more first detectable labels and one or more second detectable labels. For example, the third affinity reagent may comprise or is a protein assembly system described herein. In some embodiments, the protein assembly system used in the three-tag method comprises:
a protein (e.g., an antibody) labeled with one or more first detectable labels;
one or more biotin moieties containing linkers covalently attached to the protein; and
one or more biotin-binding proteins (e.g., avidins such as streptavidin or neutravidin) bound to the one or more biotin moieties via noncovalent interactions;
wherein the biotin-binding proteins are labeled with one or more second detectable labels, and wherein the first detectable labels are spectrally distinguishable from the second detectable labels. In further embodiments, the remaining biotin binding sites of the biotin-binding proteins (e.g., avidins such as streptavidin or neutravidin) in the protein assembly system are further blocked with a biotin moiety-containing molecule or an analog thereof such that the protein assembly system may only bind to the hapten moiety specific to the protein (e.g., the antibody).

Though certain haptens and hapten-binding partners have been discussed in relation to two-tag or three-tag sequencing schemes, other hapten and hapten-binding partners may be used, such as those exemplified in Table 1.

In any embodiments of the sequencing method described herein, the incorporation mixture further comprises a fourth type of unlabeled nucleotide, wherein the fourth type of unlabeled nucleotide is not capable of specific binding to any of the affinity reagents. Furthermore, the fourth type of unlabeled nucleotide cannot be excited by either the first or the second light source.

The term “signal state” when used in reference to an imaging event, refers to the state of a polynucleotide incorporating an unlabeled nucleotide and subsequently conjugate with a labeled affinity reagent described herein, in which a specific emission signal is produced by such imaging event, and the emission signal is measured, detected or collected. For example, one or more detectable labels of the affinity reagent specifically bound to the incorporated unlabeled nucleotide may be excited by a light source (e.g., a laser) at a specific wavelength and emits a fluorescent signal that is collected or detected in the single emission detection channel/filter, indicating a “signal state” in such imaging event and fluorescent measurement.

The term “dark state,” when used in reference to an imaging event, refers to the state of a polynucleotide incorporating an unlabeled nucleotide, and subsequently conjugate with a labeled affinity reagent described herein, in which no specific emission signal is produced by such imaging event, or no emission signal is measured, collected, and/or detected.

In one example of the method described herein, “C” nucleotide conjugate is determined by a signal state in the first fluorescent measurement and a dark state in the second fluorescent measurement; “T” nucleotide conjugate is determined by a dark state in the first fluorescent measurement and a signal state in the second fluorescent measurement; “A” nucleotide conjugate is determined by a signal state in the first fluorescent measurement and a signal state in the second fluorescent measurement; and “G” nucleotide conjugate is determined by a dark state in the first fluorescent measurement and a dark state in the second fluorescent measurement. In another example, “T” nucleotide conjugate is determined by a signal state in the first fluorescent measurement and a dark state in the second fluorescent measurement; “C” nucleotide conjugate is determined by a dark state in the first fluorescent measurement and a signal state in the second fluorescent measurement; “A” nucleotide conjugate is determined by a signal state in the first fluorescent measurement and a signal state in the second fluorescent measurement; and “G” nucleotide conjugate is determined by a dark state in the first fluorescent measurement and a dark state in the second fluorescent measurement.

In some embodiments of the sequencing method described herein, step (e) also removes the detectable labels of the incorporated nucleotides. In some such embodiments, the detectable labels and the 3′ blocking group are removed in a single step (e.g., under the same chemical reaction condition). In other embodiments, the detectable labels and the 3′ blocking group are removed in two separate steps (e.g., the detectable labels and the 3′ blocking group are removed under two separate chemical reaction conditions). In some embodiments, the detectable labels are removed by cleaving the cleavable linker in which the hapten moiety is covalent attached to the nucleotide. In some further embodiments, the method further comprises (f) washing the solid support with an aqueous wash solution.

In some embodiments, the imaging step (d) is performed by two light sources (e.g., a laser) operating at different wavelengths. In particular, one light source may have a wavelength between about 450 nm to about 460 nm, and the other light source may have a wavelength between about 510 nm to about 540 nm, or between about 520 nm to about 535 nm. As such, step (d) comprises two separate imaging events and two fluorescent measurements.

In further embodiments of the sequencing method described herein, steps (b) through (f) are performed in repeated cycles (e.g., at least 30, 50, 100, 150, 200, 250, 300, 400, or 500 times) and the method further comprises sequentially determining the sequence of at least a portion of the target polynucleotide based on the identity of each sequentially incorporated nucleotide conjugates. In some such embodiments, steps (b) through (f) are repeated at least 50 cycles.

In some embodiments of the method described herein, the incorporation of the nucleotide conjugates is performed by a mutant of 9° N polymerase, such as those disclosed in WO 2005/024010, U.S. Publication Nos. 2020/0131484 A1, 2020/0181587 A1, and U.S. Ser. Nos. 63/412,241 and 63/433,971, each of which is incorporated by reference in its entirety. Exemplary polymerases include but not limited to Pol 812, Pol 1901, Pol 1558 or Pol 963. The amino acid sequences of Pol 812, Pol 1901, Pol 1558 or Pol 963 DNA polymerases are described, for example, in U.S. Patent Publication Nos. 2020/0131484 A1 and 2020/0181587 A1.

In some embodiments of the method described herein, the one or more four different types of nucleotides in the incorporation mixture described in step (b) include nucleotide types selected from the group consisting of A, C, G, T and U, and non-natural nucleotide analogs thereof. In further embodiments, the incorporation mixture comprises four different types of nucleotide conjugates (A, C, G, and T or U), or non-natural nucleotide analogs thereof. In further embodiments, the four different types of nucleotide conjugates are dATP, dCTP, dGTP and dTTP or dUTP, or non-natural nucleotide analogs thereof. In further embodiments, each of the four types of nucleotide conjugates in the incorporation mixture contains a 3′ blocking group.

In any embodiments of the method described herein, the density of the immobilized different target polynucleotides (i.e., clusters) on the solid support is about or at least about 50,000/mm², 100,000/mm², 150,000/mm², 200,000/mm², 250,000/mm², 300,000/mm², 350,000/mm², or 400,000/mm^{2.}

Though certain haptens and hapten-binding partners have been discussed in relation to two-tag sequencing schemes, other hapten and hapten-binding partners may be used. Table 1 lists non-limiting examples of haptens and hapten-binding partners. In further embodiments, the incorporation mixture used in the sequencing method may also include one or more labeled nucleotide(s). For example, the labeled nucleotide(s) may contain a fluorescent label that cannot be excitable by either the first or the second light source.

In any embodiments of the method described herein, the first detectable label may be a blue dye described herein (e.g., the coumarin dye of Formula (I), (I-1), (I-2), (I-3) or (I-4). In some further embodiments, the second detectable label may be a green dye (e.g., a cyanine dye) described herein.

In any embodiments of methods of sequencing described herein, the method can reduce or eliminate sequence context or sequence specific effect. Two-channel base-calling relies upon the ability to discriminate bases by their intensity in two emission color channels. This intensity modulation adds noise to the system and can cause miscalls when the sequence-specific intensity modulation shift a given cluster's intensity towards a decision boundary. Using a single ffN (such as ffA) which is subsequently conjugated with a protein labeled with two colored dyes that can produce color in two channels reduce or eliminate sequence-specific intensity shifts and thereby improve base calling accuracy.

### General Description on Sequencing by Synthesis

In a specific embodiment, a synthetic step is carried out and may optionally comprise incubating a template or target polynucleotide strand with a reaction mixture comprising fluorescently labeled nucleotides of the disclosure. A polymerase can also be provided under conditions which permit formation of a phosphodiester linkage between a free 3′ hydroxy group on a polynucleotide strand annealed to the template or target polynucleotide strand and a 5′ phosphate group on the labeled nucleotide. Thus, a synthetic step can include formation of a polynucleotide strand as directed by complementary base pairing of nucleotides to a template/target strand.

In all embodiments of the methods, the detection step may be carried out while the polynucleotide strand into which the labeled nucleotides are incorporated is annealed to a template/target strand, or after a denaturation step in which the two strands are separated. Further steps, for example chemical or enzymatic reaction steps or purification steps, may be included between the synthetic step and the detection step. In particular, the polynucleotide strand incorporating the labeled nucleotide(s) may be isolated or purified and then processed further or used in a subsequent analysis. By way of example, polynucleotide strand incorporating the labeled nucleotide(s) as described herein in a synthetic step may be subsequently used as labeled probes or primers. In other embodiments, the product of the synthetic step set forth herein may be subject to further reaction steps and, if desired, the product of these subsequent steps purified or isolated.

Suitable conditions for the synthetic step will be well known to those familiar with standard molecular biology techniques. In one embodiment, a synthetic step may be analogous to a standard primer extension reaction using nucleotide precursors, including the labeled nucleotides as described herein, to form an extended polynucleotide strand (primer polynucleotide strand) complementary to the template/target strand in the presence of a suitable polymerase enzyme. In other embodiments, the synthetic step may itself form part of an amplification reaction producing a labeled double stranded amplification product comprised of annealed complementary strands derived from copying of the primer and template polynucleotide strands. Other exemplary synthetic steps include nick translation, strand displacement polymerization, random primed DNA labeling, etc. A particularly useful polymerase enzyme for a synthetic step is one that is capable of catalyzing the incorporation of the labeled nucleotides as set forth herein. A variety of naturally occurring or mutant/modified polymerases can be used. By way of example, a thermostable polymerase can be used for a synthetic reaction that is carried out using thermocycling conditions, whereas a thermostable polymerase may not be desired for isothermal primer extension reactions. Suitable thermostable polymerases which are capable of incorporating the labeled nucleotides according to the disclosure include those described in WO 2005/024010 or WO06120433, each of which is incorporated herein by reference. In synthetic reactions which are carried out at lower temperatures such as 37° C., polymerase enzymes need not necessarily be thermostable polymerases, therefore the choice of polymerase will depend on a number of factors such as reaction temperature, pH, strand-displacing activity and the like.

In specific non-limiting embodiments, the disclosure encompasses methods of nucleic acid sequencing, re-sequencing, whole genome sequencing, single nucleotide polymorphism scoring, any other application involving the detection of the modified nucleotide or nucleoside labeled with dyes set forth herein when incorporated into a polynucleotide.

SBS generally involves sequential addition of one or more nucleotides or oligonucleotides to a growing polynucleotide chain in the 5′ to 3′ direction using a polymerase or ligase in order to form an extended polynucleotide chain complementary to the template/target nucleic acid to be sequenced. The identity of the base present in one or more of the added nucleotide(s) can be determined in a detection or “imaging” step. The identity of the added base may be determined after each nucleotide incorporation step. The sequence of the template may then be inferred using conventional Watson-Crick base-pairing rules. The use of the nucleotides labeled with dyes set forth herein for determination of the identity of a single base may be useful, for example, in the scoring of single nucleotide polymorphisms, and such single base extension reactions are within the scope of this disclosure.

In an embodiment of the present disclosure, the sequence of a template/target polynucleotide is determined by detecting the incorporation of one or more nucleotides into a nascent strand complementary to the template polynucleotide to be sequenced through the detection of fluorescent label(s) attached to the incorporated nucleotide(s). Sequencing of the template polynucleotide can be primed with a suitable primer (or prepared as a hairpin construct which will contain the primer as part of the hairpin), and the nascent chain is extended in a stepwise manner by addition of nucleotides to the 3′ end of the primer in a polymerase-catalyzed reaction.

In particular embodiments, each of the different nucleotide triphosphates (A, T, G and C) may be labeled with a unique fluorophore and also comprises a blocking group at the 3′ position to prevent uncontrolled polymerization. Alternatively, one of the four nucleotides may be unlabeled (dark). The polymerase enzyme incorporates a nucleotide into the nascent chain complementary to the template/target polynucleotide, and the blocking group prevents further incorporation of nucleotides. Any unincorporated nucleotides can be washed away and the fluorescent signal from each incorporated nucleotide can be “read” optically by suitable means, such as a charge-coupled device using light source excitation and suitable emission filters. The 3′-blocking group and fluorescent dye compounds can then be removed (deprotected) (simultaneously or sequentially) to expose the nascent chain for further nucleotide incorporation.

The method, as exemplified above, utilizes the incorporation of fluorescently labeled, 3′ blocked nucleotides A, G, C, and T into a growing strand complementary to the immobilized polynucleotide, in the presence of DNA polymerase. The polymerase incorporates a base complementary to the target polynucleotide but is prevented from further addition by the 3′ blocking group. The label of the incorporated nucleotide can then be determined, and the blocking group removed by chemical cleavage to allow further polymerization to occur. The nucleic acid template to be sequenced in a SBS reaction may be any polynucleotide that it is desired to sequence. The nucleic acid template for a sequencing reaction will typically comprise a double stranded region having a free 3′ hydroxy group that serves as a primer or initiation point for the addition of further nucleotides in the sequencing reaction. The region of the template to be sequenced will overhang this free 3′ hydroxy group on the complementary strand. The overhanging region of the template to be sequenced may be single stranded but can be double-stranded, provided that a “nick” is present on the strand complementary to the template strand to be sequenced to provide a free 3′ OH group for initiation of the sequencing reaction. In such embodiments, sequencing may proceed by strand displacement. In certain embodiments, a primer bearing the free 3′ hydroxy group may be added as a separate component (e.g., a short oligonucleotide) that hybridizes to a single-stranded region of the template to be sequenced. Alternatively, the primer and the template strand to be sequenced may each form part of a partially self-complementary nucleic acid strand capable of forming an intra-molecular duplex, such as for example a hairpin loop structure. Hairpin polynucleotides and methods by which they may be attached to solid supports are disclosed in PCT Publication Nos. WO0157248 and WO2005/047301, each of which is incorporated herein by reference. Nucleotides can be added successively to a growing primer, resulting in synthesis of a polynucleotide chain in the 5′ to 3′ direction. The nature of the base which has been added may be determined, particularly but not necessarily after each nucleotide addition, thus providing sequence information for the nucleic acid template. Thus, a nucleotide is incorporated into a nucleic acid strand (or polynucleotide) by joining of the nucleotide to the free 3′ hydroxy group of the nucleic acid strand via formation of a phosphodiester linkage with the 5′ phosphate group of the nucleotide.

The nucleic acid template to be sequenced may be DNA or RNA, or even a hybrid molecule comprised of deoxynucleotides and ribonucleotides. The nucleic acid template may comprise naturally occurring and/or non-naturally occurring nucleotides and natural or non-natural backbone linkages, provided that these do not prevent copying of the template in the sequencing reaction.

In certain embodiments, the nucleic acid template to be sequenced may be attached to a solid support via any suitable linkage method known in the art, for example via covalent attachment. In certain embodiments template polynucleotides may be attached directly to a solid support (e.g., a silica-based support). However, in other embodiments of the disclosure the surface of the solid support may be modified in some way so as to allow either direct covalent attachment of template polynucleotides, or to immobilize the template polynucleotides through a hydrogel or polyelectrolyte multilayer, which may itself be non-covalently attached to the solid support.

Arrays in which polynucleotides have been directly attached to a support (for example, silica-based supports such as those disclosed in WO00/06770 (incorporated herein by reference), wherein polynucleotides are immobilized on a glass support by reaction between a pendant epoxide group on the glass with an internal amino group on the polynucleotide. In addition, polynucleotides can be attached to a solid support by reaction of a sulfur-based nucleophile with the solid support, for example, as described in WO2005/047301 (incorporated herein by reference). A still further example of solid-supported template polynucleotides is where the template polynucleotides are attached to hydrogel supported upon silica-based or other solid supports, for example, as described in WO00/31148, WO01/01143, WO02/12566, WO03/014392, U.S. Pat. No. 6,465,178 and WO00/53812, each of which is incorporated herein by reference.

A particular surface to which template polynucleotides may be immobilized is a polyacrylamide hydrogel. Polyacrylamide hydrogels are described in the references cited above and in WO2005/065814, which is incorporated herein by reference. Specific hydrogels that may be used include those described in WO2005/065814 and U.S. Pub. No. 2014/0079923. In one embodiment, the hydrogel is PAZAM (poly(N-(5-azidoacetamidylpentyl) acrylamide-co-acrylamide)).

DNA template molecules can be attached to beads or microparticles, for example, as described in U.S. Pat. No. 6,172,218 (which is incorporated herein by reference). Attachment to beads or microparticles can be useful for sequencing applications. Bead libraries can be prepared where each bead contains different DNA sequences. Exemplary libraries and methods for their creation are described in Nature, 437, 376-380 (2005); Science, 309, 5741, 1728-1732 (2005), each of which is incorporated herein by reference. Sequencing of arrays of such beads using nucleotides set forth herein is within the scope of the disclosure.

Template(s) that are to be sequenced may form part of an “array” on a solid support, in which case the array may take any convenient form. Thus, the method of the disclosure is applicable to all types of high-density arrays, including single-molecule arrays, clustered arrays, and bead arrays. Nucleotides labeled with dye compounds of the present disclosure may be used for sequencing templates on essentially any type of array, including but not limited to those formed by immobilization of nucleic acid molecules on a solid support.

However, nucleotides labeled with dye compounds of the disclosure are particularly advantageous in the context of sequencing of clustered arrays. In clustered arrays, distinct regions on the array (often referred to as sites, or features) comprise multiple polynucleotide template molecules. Generally, the multiple polynucleotide molecules are not individually resolvable by optical means and are instead detected as an ensemble. Depending on how the array is formed, each site on the array may comprise multiple copies of one individual polynucleotide molecule (e.g., the site is homogenous for a particular single- or double-stranded nucleic acid species) or even multiple copies of a small number of different polynucleotide molecules (e.g., multiple copies of two different nucleic acid species). Clustered arrays of nucleic acid molecules may be produced using techniques generally known in the art. By way of example, WO 98/44151 and WO00/18957, each of which is incorporated herein, describe methods of amplification of nucleic acids wherein both the template and amplification products remain immobilized on a solid support in order to form arrays comprised of clusters or “colonies” of immobilized nucleic acid molecules. The nucleic acid molecules present on the clustered arrays prepared according to these methods are suitable templates for sequencing using nucleotides labeled with dye compounds of the disclosure.

Nucleotides labeled with dye compounds of the present disclosure are also useful in sequencing of templates on single molecule arrays. The term “single molecule array” or “SMA” as used herein refers to a population of polynucleotide molecules, distributed (or arrayed) over a solid support, wherein the spacing of any individual polynucleotide from all others of the population is such that it is possible to individually resolve the individual polynucleotide molecules. The target nucleic acid molecules immobilized onto the surface of the solid support can thus be capable of being resolved by optical means in some embodiments. This means that one or more distinct signals, each representing one polynucleotide, will occur within the resolvable area of the particular imaging device used.

Single molecule detection may be achieved wherein the spacing between adjacent polynucleotide molecules on an array is at least 100 nm, more particularly at least 250 nm, still more particularly at least 300 nm, even more particularly at least 350 nm. Thus, each molecule is individually resolvable and detectable as a single molecule fluorescent point, and fluorescence from said single molecule fluorescent point also exhibits single step photobleaching.

The terms “individually resolved” and “individual resolution” are used herein to specify that, when visualized, it is possible to distinguish one molecule on the array from its neighboring molecules. Separation between individual molecules on the array will be determined, in part, by the particular technique used to resolve the individual molecules. The general features of single molecule arrays will be understood by reference to published applications WO00/06770 and WO 01/57248, each of which is incorporated herein by reference. Although one use of the labeled nucleotides of the disclosure is in SBS reactions, the utility of such nucleotides is not limited to such methods. In fact, the labeled nucleotides described herein may be used advantageously in any sequencing methodology which requires detection of fluorescent labels attached to nucleotides incorporated into a polynucleotide.

In particular, nucleotides labeled with dye compounds of the disclosure may be used in automated fluorescent sequencing protocols, particularly fluorescent dye-terminator cycle sequencing based on the chain termination sequencing method of Sanger and co-workers. Such methods generally use enzymes and cycle sequencing to incorporate fluorescently labeled dideoxynucleotides in a primer extension sequencing reaction. So-called Sanger sequencing methods, and related protocols (Sanger-type), utilize randomized chain termination with labeled dideoxynucleotides.

### EXAMPLES

Additional embodiments are disclosed in further detail in the following examples, which are not in any way intended to limit the scope of the claims.

### General Procedure for Determining Degree of Protein Labeling

The degree of protein labeling by a dye may be estimated via absorbance measurements. First, if present, nonconjugated dye must be removed from a sample including dye-labeled protein. The nonconjugated dye should be completely, or substantially removed from the sample for accurate determination of the protein:dye ratio. The dye may be removed by dialysis or gel filtration, for example.

After removing nonconjugated dye, the absorbance of the protein:dye conjugate may be measured at a wavelength of 280 nm, A₂₈₀. The 280 nm absorbance A₂₈₀ may be measured using a spectrophotometer. The cuvette of the spectrophotometer may have a 1 cm path length. If an initial absorbance measurement exceeds 2.0, the sample may be diluted by a dilution factor DF necessary to lower the absorbance below 2.0 before remeasuring. Additionally, an absorbance Aₘₐₓ of the protein:dye conjugate may be measured at the wavelength λₘₐₓ at which the dye is maximally absorbent.

Molarity of the protein in the sample may be calculated according to the following:

$$protein concentration ( M ) = \frac{{A}_{280} - ( {A}_{max} \times CF )}{ε} \times DF$$

wherein ε is the protein molar extinction coefficient and CF is the correction factor that adjusts for the amount of absorbance at 280 nm caused by the dye. The molar protein concentration can be used to calculate the protein:dye molar ratio:

$$dye per mole protein = \frac{{A}_{max} of the labeled protein}{{ε}^{′} \times [ protein concentration ( M ) ]} \times DF$$

wherein ε′ is the molar extinction coefficient of the fluorescent dye.

### Example 1. Preparation of Blue and Green Dyes Labeled Streptavidin

To prepare blue dye-labeled streptavidin, 1 equivalent of coumarin dye A-PEG₁₂-COOH was co-evaporated with 1 mL of DMF and dried under vacuum. Then, 10 equivalents of N,N-diisopropylethylamine and 1.1 equivalents of TSTU were added. The reaction was stirred at room temperature for 30 min to form the activated ester of the coumarin dye A-PEG₁₂-COOH. Then 10 μL of the activated ester was added to 1.0 mL of 2.0 mg/mL streptavidin (SA) in pH 6.5 potassium phosphate buffer. The resulting solution was stirred at room temperature for 1 hour and the reaction mixture was purified by a PD minitrap G-25 size exclusion column using pH 6.5, 10 mM potassium phosphate buffer to afford a resulting streptavidin (coumarin A-PEG₁₂) conjugate. Coumarin dye A was covalently attached to streptavidin through the reaction of the activated ester of the carboxyl group and the lysine residue of the streptavidin to form amide bonding. The number of coumarin dye A covalently attached to streptavidin was calculated based on the general procedure for determining degree of protein labeling by compare the ratio of absorption wavelength of protein and dye. The resulting protein dye conjugate was determined as SA(coumarin dye A-PEG₁₂)_{4.5}.

To prepare green dye labeled streptavidin, 1 equivalent of cyanine dye B was co-evaporated with 1 mL of DMF and dried under vacuum. Then 10 equivalent of N, N-Diisopropylethylamine and 1.1 equivalent of TSTU were added. The reaction was stirred at room temperature for 30 min to form the activated ester of the cyanine dye B. The resulting conjugate 5.0 μM SA(coumarin dye A-PEG₁₂)45 in pH 6.5 potassium phosphate was then mixed with 10 μL of activated ester of cyanine dye B and stirred at room temperature for 1 hour and the reaction mixture was purified by a PD minitrap G-25 size exclusion columns using pH 8.0, 10 mM tris buffer to obtain dual labeled streptavidin (labeled with both blue coumarin dye A and green cyanine dye B). The degree of cyanine dye B labeling was calculated based on the same general procedure as described above as SA(coumarin dye A-PEG₁₂)_{4.5}(cyanine dye B)_{1.4}. The complex was characterized by nanodrop photo spectrometer.

Coumarin dye A is disclosed in U.S. Publication No. 2022/0033900 A1, having the structure moiety

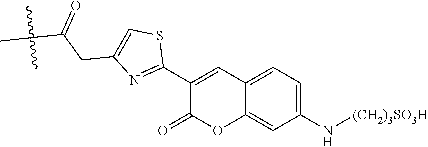

when conjugated with a protein (e.g., streptavidin) or antibody (e.g., anti-DNP) through the PEG linker. This coumarin dye is excitable by a blue light at about 450 nm to about 460 nm (also called a “blue dye”). Cyanine dye B is disclosed in International Publication No. WO2014/135221 A1, having the structure moiety

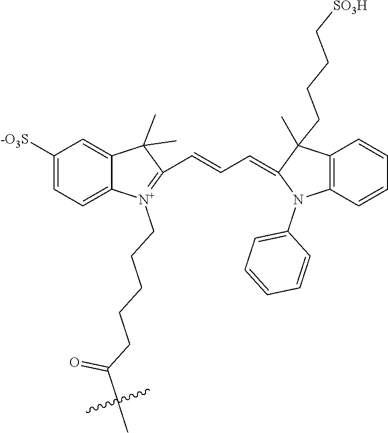

when conjugated with streptavidin. This cyanine dye is excitable by a green light at about 520 nm to about 540 nm (also called a “green dye”).

### Example 2. Preparation of a dual-protein assembly system

First, 1 equivalent of coumarin dye A-PEG₁₂-COOH was co-evaporated with 1 mL of DMF and dried under vacuum. Then 10 equivalent of N, N-diisopropylethylamine and 1.1 equivalent of TSTU were added. The reaction was stirred at room temperature for 30 min to form the activated ester of the coumarin dye A-PEG₁₂-COOH. Then 20 μL of the activated ester of the coumarin dye A-PEG₁₂-COOH was added to 0.5 mL of 2.0 mg/mL (12.5 anti-DNP antibody in pH 6.5 potassium phosphate buffer. The resulting solution was stirred at room temperature for 1 hour and the reaction mixture was purified by a PD minitrap G-25 size exclusion column using pH 6.5, 10 mM potassium phosphate buffer to obtain coumarin dye A labeled anti-DNP. The degree of dye labeling was calculated based on the general procedure described above as anti-DNP(coumarin dye A-PEG₁₂)₇. Then, 1 mL of 6.2 μM anti-DNP(coumarin dye A-PEG₁₂) 7 was mixed with 10 μL of 2.35 mg/mL of NHS-PEG₁₂-Biotin in DMA solution and stirred at room temperature for 1 hour and the reaction mixture was purified by a PD minitrap G-25 size exclusion column using pH 8.0, 10 mM tris buffer to obtain anti-DNP(coumarin dye A-PEG₁₂) 7 -Biotin with 3.0 μM solution concentration.

The preparation of cyanine dye B labeled streptavidin SA(cyanine dye B)₇ was similar to the procedure described in U.S. Publication No. 2013/0079232 A1, which is incorporated by reference in its entirety. The degree of dye labeling was calculated based on the general procedure described above as SA(cyanine dye B)₇. The dual protein system was then assembled by adding 48 of 3.0 μM of anti-DNP(coumarin dye A-PEG₁₂)₇ -Biotin and 31.5 μL of 13.7 μM SA(cyanine dye B)₇ (3 molar equivalent) in 20.5 μL of pH 8.0 10 mM tris buffer to form anti-DNP(coumarin dye A-PEG₁₂)₇ -Biotin-[SA(cyanine dye B)₇]₃. The dual protein complex was characterized by nanodrop photo spectrometer.

### Example 3. Two-Tag SBS with Unlabeled Nucleotides

In this example, SBS was conducted using a two-dye scheme with universal ffNs having two different hapten moieties. The unlabeled ffNs used in the incorporation mix included dark ffG, biotin-linked ffA (ffA-sPA-LN3-biotin), DNP-linked ffA (ffA-sPA-LN3-DNP), DNP-linked ffT (ffT-LN3-DNP), biotin-linked ffC (ffC-LN3-LN3-biotin), and S07181-linked ffC (ffC-LN3-SO7181). SO7181 is a commercially available red dye. An affinity reagent mixture used in the secondary labeling after the incorporation of the unlabeled nucleotides included 0.1 μM anti-DNP antibody labeled with coumarin dye A (anti-DNP(coumarin dye A)₄), and 0.1 μM of streptavidin labeled with cyanine dye B. The affinity reagent mixture was introduced to incorporated ffNs and allowed to bind at 60° C. for 60 seconds. The SBS run was conducted on a MiSeg™ using blue/green light excitations (first blue light at about 460 nm, then green light at about 532 nm).

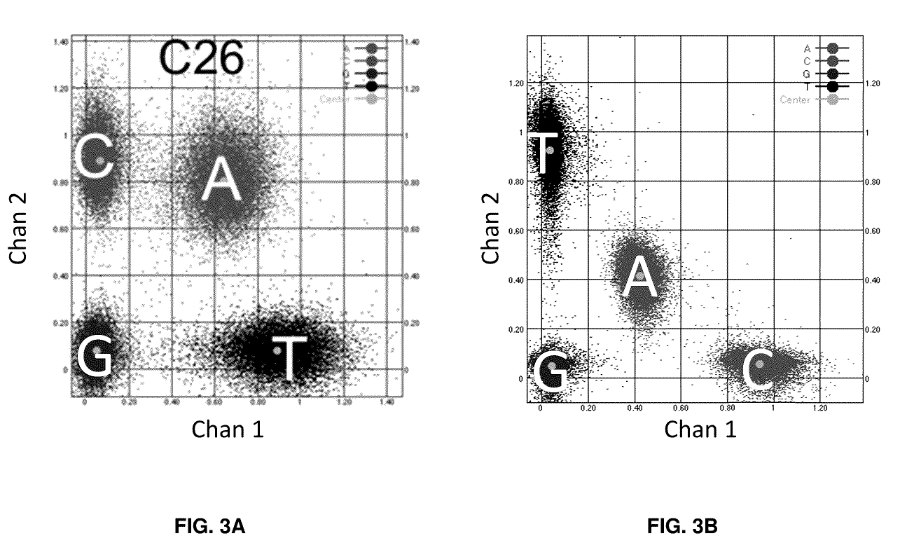

FIG. 3A is a two-dye SBS signal intensity scatterplot generated from a reading at incorporation cycle 26 of that SBS run. Channel 1 (x-axis) plots intensity in the green channel while Channel 2 (y-axis) plots intensity in the blue channel. For that run, the average error rate over 150 cycles was 1.1%, the average phasing was 0.07%, and the average signal remaining was 61%.

A second SBS run was conducted under similar conditions with reversed labeled protein tags. It included 0.1 μM anti-DNP antibody labeled with cyanine dye B, and 0.1 μM of streptavidin labeled with coumarin dye A. FIG. 3B is a two-dye SBS intensity plot generated from a reading at incorporation cycle 26 of that run. Channel 1 (x-axis) plots intensity in the blue channel while Channel 2 (y-axis) plots intensity in the green channel. For this run, the error rate was 0.54%, the phasing was 0.13%, and the signal remaining was 63%.

### Example 4. Three-Tag SBS with Unlabeled Nucleotides

In this example, SBS was conducted using a three-tag system with a set of unlabeled nucleotides having three different hapten moieties. The ffNs used included dark ffG, DNP-linked ffA (ffA-sPA-LN3-DNP), biotin-linked ffC (ffC-LN3-LN3-biotin), and DIG-linked ffT (ffT-sPA-LN3-DIG). The affinity reagent mixture (i.e., labeled protein mixture) included a dual-labeled streptavidin complex SA(coumarin dye A-PEG₁₂)_{4.5}(cyanine dye B)_{1.4} as described in Example 1, including, on average, 4.5 molar equivalent coumarin dye A moieties (blue dye) and 1.4 molar equivalent cyanine dye B (green dye); an anti-DIG antibody labeled with cyanine dye B (anti-DIG(cyanine dye B)₈) and an anti-DNP antibody labeled with coumarin dye A (anti-DNP(coumarin dye A)₄). The labeling protein mix was introduced to incorporated ffNs and allowed to bind at 60° C. for 60 seconds. The SBS run was conducted on a MiSegTM using blue/green light excitations.

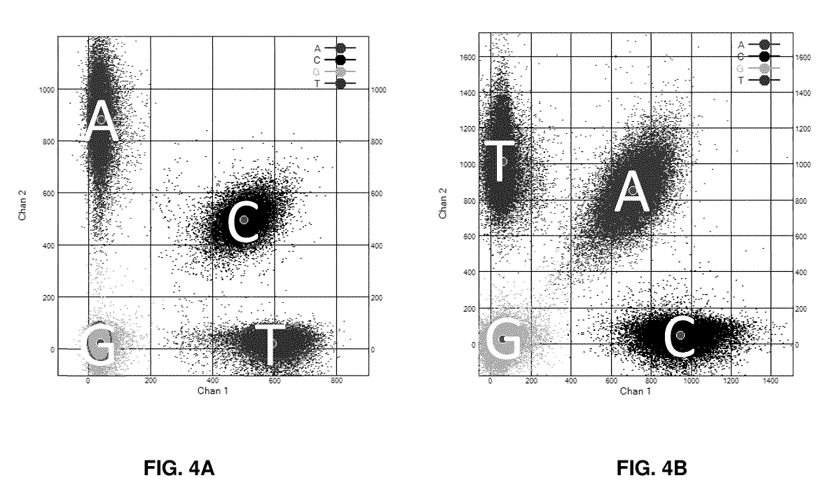

FIG. 4A is a two-dye SBS signal intensity scatterplot generated from incorporation cycle 1 of that run. Channel 1 (x-axis) plots intensity in the green channel while Channel 2 (y-axis) plots intensity in the blue channel. For that run, error rate at cycle 150 was 0.79%, phasing was 0.059%, and signal remaining was 86%.

A second SBS run with a different affinity reagent mixture was conducted under similar conditions. The ffNs in the incorporation mixture included: dark ffG, DNP-linked ffA (ffA-sPA-LN3-DNP), biotin-linked ffC (ffC-LN3-LN3-biotin), and DIG-linked ffT (ffT-sPA-LN3-DIG). The affinity reagent mixture (i.e., labeled protein mixture) included a dual protein assembly system described in Example 2— anti-DNP(coumarin dye A-PEG₁₂)₇ -Biotin-[SA(cyanine dye B)₇]₃. The anti-DNP antibody was covalently labeled with coumarin dye A moieties (blue dye). In addition, the anti-DNP antibody was linked via biotin linker to three molar equivalent of cyanine dye B (green dye) labeled streptavidin. These streptavidin molecules were on average labeled with seven cyanine dye B moieties and any remaining biotin-binding sites were blocked such that this protein assembly cannot bind to biotin-linked ffC. The labeling protein mix also included an anti-DIG antibody labeled with cyanine dye B (anti-DIG(cyanine dye B)₈) and streptavidin labeled with coumarin dye A (SA(coumarin dye A)₄).

FIG. 4B is a two-dye SBS signal intensity scatterplot generated from incorporation cycle 1 of that second run. Channel 1 (x-axis) plots intensity in the blue channel while Channel 2 (y-axis) plots intensity in the green channel. Furthermore, it was observed that there was no Channel 1-Channel 2 anticorrelated sequence context behavior of the A cloud (typically observed in two-channel SBS when ffA is labeled with either a blue or a green dye) in the scatterplot because of the single entity labels for each of the ffA, ffC and ffT.

### Example 5. Synthesis of Hydrophilic Coumarin Dyes of Formula (I) and Fluorescent Intensity Solution Data

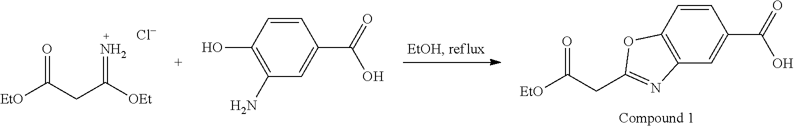 [SMILES: CCOC(=N)CC(=O)OCC ; [Cl-] ; Nc1cc(C(=O)O)ccc1O ; CCOC(=O)Cc1nc2cc(C(=O)O)ccc2o1]

3-Amino-4-hydroxybenzoic acid (4.05 g, 26.5 mmol, 1.0 eq) was suspended in EtOH (50 ml) and cooled to 0° C. Ethyl 3-ethoxy-3-iminopropionate hydrochloride (5.69 g, 29.1 mmol, 1.1 eq) was added and the flask allowed to warm to RT, followed by heating under reflux for 16 hrs. The reaction mixture was cooled to RT and the precipitate formed collected by filtration, washing with EtOH. The target Compound 1 was collected as an off-white powder (5.99 g, 24.1 mmol, 91%). 1H NMR (400 MHz, DMSO) δ 13.12 (s, <1H), 8.26 (d, J=1.6 Hz, 1H), 8.03 (dd, J=8.5, 1.7 Hz, 1H), 7.84 (d, J=8.5 Hz, 1H), 4.27 (s, 2H), 4.17 (q, J=7.1 Hz, 2H), 1.21 (t, J=7.1 Hz, 3H).

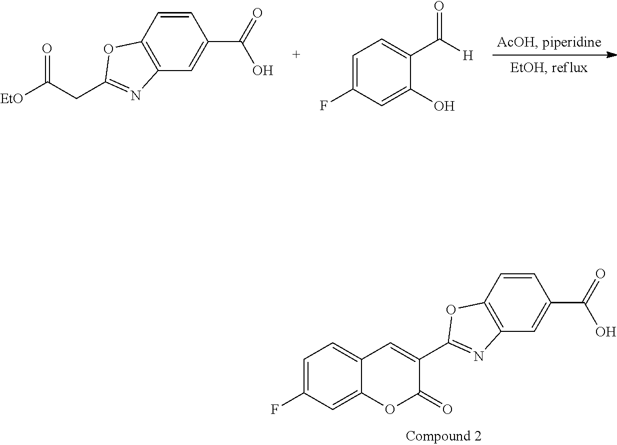 [SMILES: CCOC(=O)Cc1nc2cc(C(=O)O)ccc2o1 ; O=Cc1ccc(F)cc1O ; O=C(O)c1ccc2oc(-c3cc4ccc(F)cc4oc3=O)nc2c1]

4-Fluoro-2-hydroxybenzaldehyde (3.35 g, 23.9 mmol, 1.0 eq) and Compound 1 (5.97 g, 23.9 mmol, 1.0 eq) were suspended in EtOH, followed by addition of AcOH (836 μl, 12.0 mmol, 0.5 eq) and piperidine (974 μl, 12.0 mmol, 0.5 eq). The reaction was heated under reflux for 16 hrs (orange suspension formed which dissolved on heating). The reaction mixture was cooled to RT and the precipitate collected by filtration, washed with EtOH and dried under high vacuum to afford the Compound 2 as a pale-yellow solid (5.05 g, 15.5 mmol, 65%). 1H NMR (400 MHz, DMSO) δ 9.13 (s, 1H), 8.29 (d, J=1.5 Hz, 1H), 8.15-8.03 (m, 2H), 7.80 (d, J=8.5 Hz, 1H), 7.52 (dd, J=9.6, 2.5 Hz, 1H), 7.38 (td, J=8.8, 2.5 Hz, 1H).

### Synthesis of Dye I-1

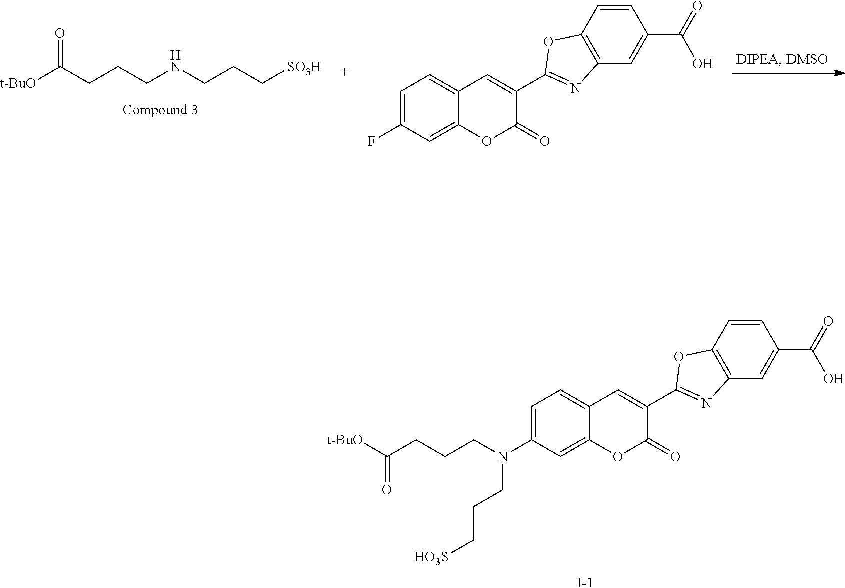 [SMILES: CC(C)(C)OC(=O)CCCNCCCS(=O)(=O)O ; O=C(O)c1ccc2oc(-c3cc4ccc(F)cc4oc3=O)nc2c1 ; CC(C)(C)OC(=O)CCCN(CCCS(=O)(=O)O)c1ccc2cc(-c3nc4cc(C(=O)O)ccc4o3)c(=O)oc2c1 ; [I-]]

Compound 3 (53.5 mg, 190 mol, 2.0 eq) and Compound 2 (30.9 mg, 95 μmol, 1.0 eq) were suspended in DMSO (2 ml). DIPEA (165 μl, 950 mol, 10.0 eq) was added and the reaction heated at 110° C. for 16 hrs. (Orange suspension to dark orange solution). Half the volume of solvent was distilled off under high vacuum at 60° C. and the residue diluted with a 1:1 mixture of water and acetonitrile (10 ml). The product was purified by Prep HPLC (eluting at 35% acetonitrile/water) to afford Dye I-1 (31.3 mol, 33%). λ_{abs}=450 nm, λ_{fl}=495 nm.

### Synthesis of Dye I-2

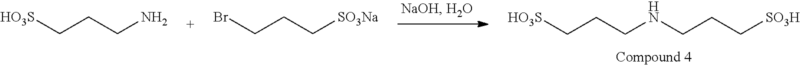

A solution of 3-amino-1-propane sulfonic acid (0.75 g, 5.39 mmol, 1.0 eq) in water (5 ml) was heated to 60° C. Aqueous NaOH (2 M, 3 ml) was added, followed by a solution of 3-bromopropane sulfonic acid sodium salt (1.40 g, 6.22 mmol, 1.15 eq) in water (5 ml) dropwise and stirred at 60° C. for 16 hrs. The reaction mixture was evaporated and redissolved in a minimum volume of water and purified by C₁₈ column chromatography (0-10% Acetonitrile/Water) to give Compound 4 as a crude mixture with undesired primary amine and tertiary amine (unreactive in next step).

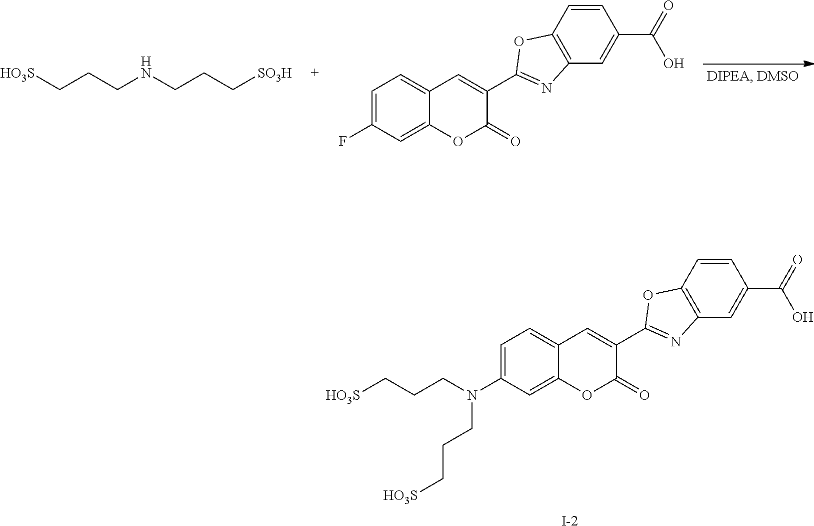

Compound 4 (2.0 g crude mixture) and Compound 2 (750 mg, 2.31 mmol, 1.0 eq) were suspended in DMSO (40 ml). DIPEA (4.0 ml, 23.1 mmol, 10.0 eq) was added and the reaction heated at 110° C. for 16 hrs. (Orange suspension to dark orange solution). Half the volume of solvent was distilled off under high vac at 60° C. and the residue diluted with water (40 ml) and filtered. Dye I-2 was purified by Prep HPLC (eluting at 20% acetonitrile/water) to afford dye I-2 (187 mol, 8%). λ_{abs}=449 nm, λ_{fl}=494 nm.

### General Synthesis of Amine-modified Coumarin Dyes

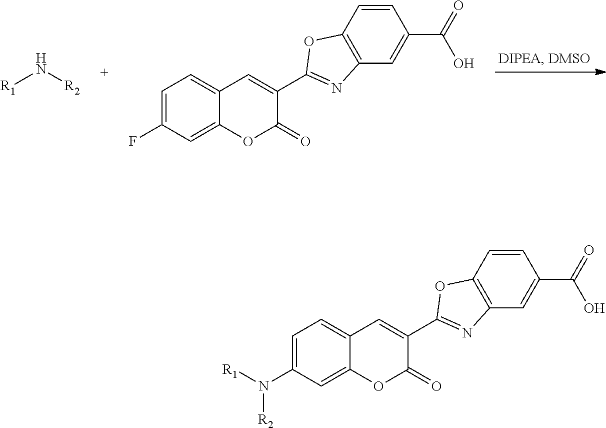

Amine R₁—NH—R₂ (2.0 eq) and Compound 2 (1.0 eq) were suspended in DMSO. DIPEA (10.0 eq) was added, and the reaction heated at 110° C. for 6-16 hrs. (Orange suspension to dark orange solution). Half the volume of solvent was distilled off under high vac at 60° C. and the residue diluted with water and filtered. The product was purified by Prep HPLC (eluting at 20-40% acetonitrile/water) to afford amine-modified coumarin dyes (8-40% yield). Using the general synthetic method described herein, dye I-4 was prepared with 40% yield. λ_{abs}=451 nm, λ_{fl}=500 nm.

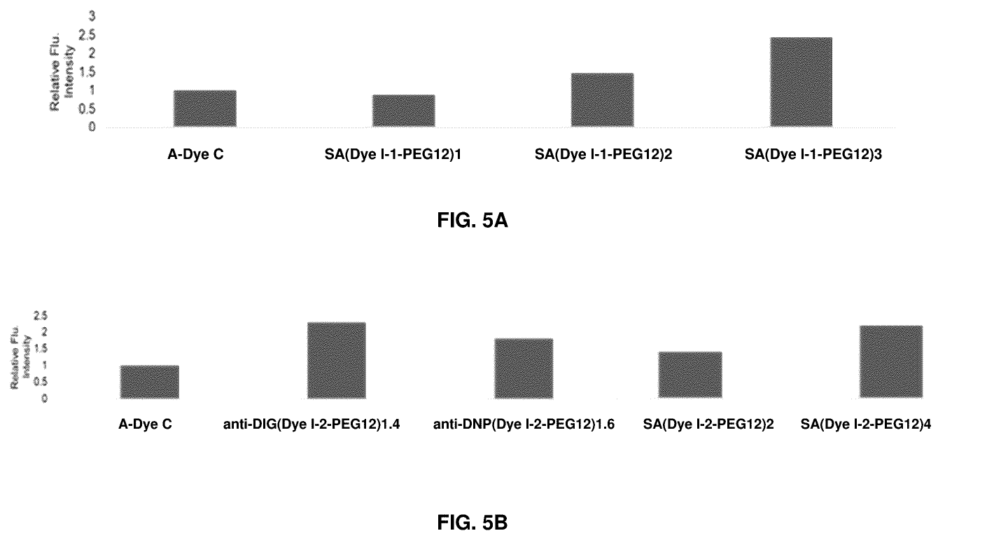

The fluorescent intensity of dye I-1 and dye I-2 were tested in solution against the reference coumarin dye C, which is a standard blue dye used in Illumina's two-channel SBS with good stability and fluorescent intensity. In addition, protein/antibody (such as streptavidin, anti-DIG, anti-DNP) labeled with multiple dye I-1 and dye I-2 were also prepared following similar procedure described in Example 1, and their fluorescent intensities were also tested in solution. As shown in FIGS. 5A and 5B, labeled streptavidin, labeled anti-DIG, and labeled anti-DNP demonstrated greater fluorescent intensities in solution compared with ffA labeled with coumarin dye C at the same concentration.

### Example 6. MiSeq® SBS Using Hydrophilic Coumarin Dyes of Formula (I)

ffC-sPA-dye I-1 and ffC-sPA-dye I-2 were tested on Illumina's MiSeg™ platform with blue and green LED operating at 460 nm and 532 nm, against the standard ffC-sPA-coumarin dye C. Sequencing recipe was using 22° C. scanning, no variable dosage, no re-use, 25 s incorporation time and 10 s cleavage time. In addition to the labeled ffC, additional components of the incorporation mix include: (1) a set of nucleotides comprising dark G, ffC-SO7181, ffA-LN3-BL-coumarin dye C, ffA-BL-NR550S0 (a known green dye), ffT-LN3-cyanine dye B; (2) DNA polymerase Pol 1901; and (3) a glycine buffer.

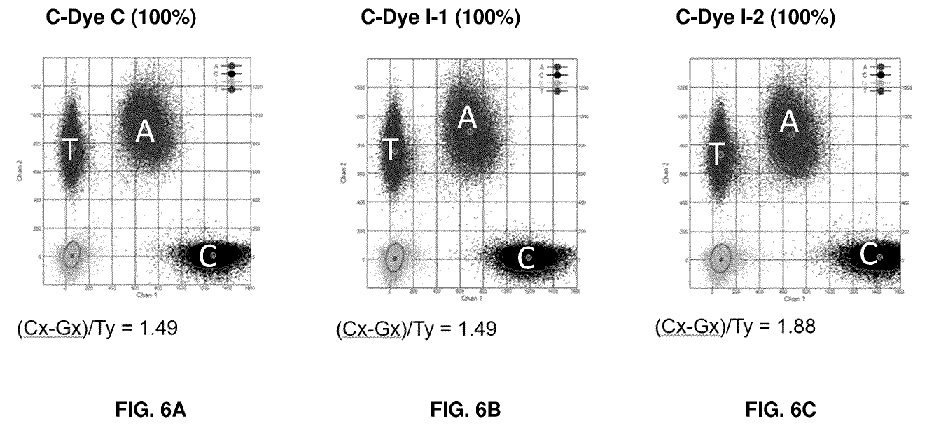

FIGS. 6A-6C are scatterplot of blue/green two-channel MiSeq® SBS runs at cycle 5, comparing the effect of changing ffC-sPA-coumarin dye C with ffC-sPA-dye I-1 or ffC-sPA-dye I-2. Both ffC-sPA-dye I-1 or ffC-sPA-dye I-2 showed an increase in brightness in sequencing compared to the standard ffC labeled with coumarin dye C.

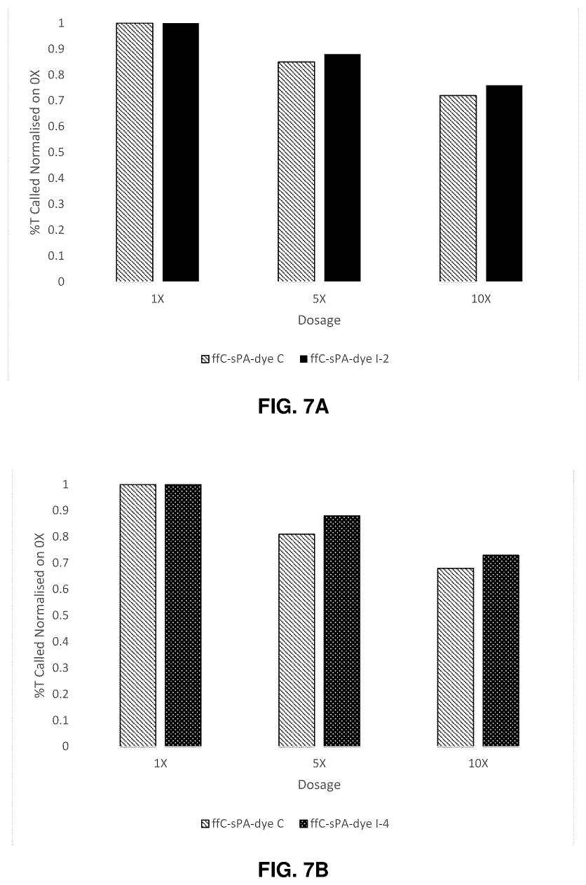

When exposed to increased dosage during sequencing, ffN mixes incorporating ffC-sPA-dye I-2 and ffC-spA-dye I-4, both retained more signal than corresponding ffC-spA-coumarin dye C (FIGS. 7A and 7B). This may indicate that the growing DNA strand was damaged less by these ffNs during each cycle of the sequencing process.

1. A kit for sequencing application, comprising:
a first type of unlabeled nucleotide comprising a first hapten;
a second type of unlabeled nucleotide comprising a second hapten;
a third type of unlabeled nucleotide; and
a set of affinity reagents comprising:
a first affinity reagent comprising a first hapten-binding partner that is capable of specific binding to the first type of unlabeled nucleotide; and
a second affinity reagent comprising a second hapten-binding partner that is capable of specific binding to the second type of unlabeled nucleotide;
wherein the first affinity reagent comprises one or more first detectable labels that are excitable by a first excitation light source, the second affinity reagent comprises one or more second detectable labels that are excitable by a second excitation light source, and wherein the first detectable label is spectrally distinguishable from the second detectable label; and
wherein one of the first excitation light source and the second excitation light source has a wavelength of about 450 nm to about 460 nm, and the other one of the first excitation light source and the second excitation light source has a wavelength of about 520 nm to about 540 nm.

2. The kit of claim 1, wherein the first hapten is covalently attached to the nucleobase of the first type of unlabeled nucleotide via a cleavable linker.

3. The kit of claim 1, wherein the second hapten is covalently attached to the nucleobase of the second type of unlabeled nucleotide via a cleavable linker.

4. The kit of claim 1, wherein the third type of unlabeled nucleotide comprises a mixture of the third type of unlabeled nucleotide comprising the first hapten and the third type of unlabeled nucleotide comprising the second hapten, and wherein both the first affinity reagent and the second affinity reagent are capable of specific binding to the third type of unlabeled nucleotide.

5. The kit of claim 4, wherein each of the first hapten and the second hapten is covalently attached to the nucleobase of the third type of nucleotide via a cleavable linker.

6. The kit of claim 4, wherein the first hapten comprises a biotin moiety and the first hapten-binding partner comprises an avidin selected from streptavidin or neutravidin.

7. (canceled)

8. The kit of claim 6, wherein one or more biotin binding sites of the avidin are blocked by a biotin moiety-containing molecule or an analog thereof.

9. The kit of claim 6, wherein the second hapten comprises a dinitrophenyl (DNP) moiety and the second hapten-binding partner comprises an anti-DNP antibody. (Original) The kit of claim 9, wherein the third type of unlabeled nucleotide comprises a mixture of a third type of unlabeled nucleotide comprising a biotin moiety, and a third type of unlabeled nucleotide comprising a DNP moiety.

11. The kit of claim 6, wherein the second hapten comprises a digoxigenin (DIG) moiety and the second hapten-binding partner comprises an anti-DIG antibody.

12. The kit of claim 11, wherein the third type of unlabeled nucleotide comprises a mixture of a third type of unlabeled nucleotide comprising a biotin moiety, and a third type of unlabeled nucleotide comprising a DIG moiety.

13. The kit claim 1, wherein the third type of unlabeled nucleotide comprises a third hapten, and the set of affinity reagents further comprises a third affinity reagent comprising a third hapten-binding partner that is capable of specific binding to the third hapten.

14. The kit of claim 13, wherein the third hapten is covalently attached to the nucleobase of the third type of unlabeled nucleotide via a cleavable linker.

15. The kit of claim 13, wherein the first hapten comprises a biotin moiety and the first hapten-binding partner comprises an avidin selected from streptavidin or neutravidin.

16. (canceled)

17. The kit of claim 15, wherein one or more biotin binding sites of the avidin are blocked by a biotin moiety-containing molecule or an analog thereof.

18. The kit of claim 15, wherein one of the second hapten and the third hapten comprises a DNP moiety, and the other one of the second hapten and the third hapten comprises a DIG moiety.

19. (canceled)

20. (canceled)

21. (canceled)

22. (canceled)

23. The kit of any one of claims 13, wherein the third affinity reagent comprises one or more first detectable labels and one or more second detectable labels.

24. The kit of claim 23, wherein the third affinity reagent comprises a multi-dye labeled protein assembly system, wherein the multi-dve labeled protein assembly system comprises:
a first protein labeled with one or more first detectable labels;
one or more hapten-containing linkers covalently attached to the first protein; and
one or more hapten-binding second proteins bound to one or more hapten-containing linkers:
wherein the one or more hapten-binding second proteins are labeled with one or more second detectable labels, and wherein the one or more first detectable labels are spectrally distinguishable from the one or more second detectable labels.

25. The kit of claim 1, further comprises a fourth type of unlabeled nucleotide, wherein the fourth type of unlabeled nucleotide is not capable of specific binding to any of the affinity reagents.

26. The kit of claim 25, wherein each of the first type, second type, third type and fourth type of unlabeled nucleotide comprises a 3′ blocking group.

27. The kit of claim 1, wherein the first detectable label is a dye having the structure of Formula (I): 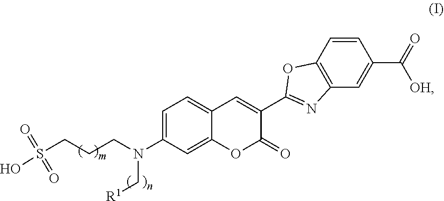
wherein
R¹ is —C(O)OC₁-C₆ alkyl, —SO₃H, —OH, optionally substituted 5 to 10 membered heteroaryl, optionally substituted 3 to 10 membered heterocyclyl, optionally substituted phenyl, or —OR^{x};
R^{x} is optionally substituted 5 to 10 membered heteroaryl, optionally substituted 3 to 10 membered heterocyclyl, or optionally substituted phenyl;
m is an integer of 1, 2 or 3; and
n is an integer of 1, 2, 3, 4 or 5.

28. The kit of claim 1, further comprising a DNA polymerase and one or more buffer compositions.

29. A protein assembly system, comprising:
a first protein labeled with one or more first detectable labels;
one or more hapten-containing linkers covalently attached to the first protein; and
one or more hapten-binding second proteins bound to one or more hapten-containing linkers;
wherein the one or more hapten-binding second proteins are labeled with one or more second detectable labels, and wherein the one or more first detectable labels are spectrally distinguishable from the one or more second detectable labels.

30-43. (canceled)

44. A nucleotide conjugate comprising the protein assembly system of claim 29.

45. (canceled)

46. (canceled)

47. (canceled)

48. An oligonucleotide or polynucleotide comprising the nucleotide conjugate of claim 44 incorporated therein.

49. (canceled)

50. (canceled)

51. A method of determining the sequences of a plurality of different target polynucleotides, comprising:
(a) contacting a solid support with a solution comprising sequencing primers under hybridization conditions, wherein the solid support comprises a plurality of different target polynucleotides immobilized thereon; and the sequencing primers are complementary to at least a portion of the target polynucleotides;
(b) contacting the solid support with an aqueous solution comprising DNA polymerase and one or more of four different types of unlabeled nucleotides under conditions suitable for DNA polymerase-mediated primer extension, and incorporating one type of nucleotides into the sequencing primers to produce extended copy polynucleotides; wherein
each of the four types of nucleotides comprises a 3′ blocking group;
the first type of unlabeled nucleotide comprises a first hapten;
the second type of unlabeled nucleotide comprises a second hapten;
(c) contacting the extended copy polynucleotides with a set of affinity reagents under conditions wherein an affinity reagent binds specifically to the incorporated unlabeled nucleotides to provide labeled extended copy polynucleotides;
(d) imaging the solid support and performing one or more fluorescent measurements of the extended copy polynucleotides; and
(e) removing the 3′ blocking group of the incorporated nucleotides;
wherein the set of affinity reagents comprises:
a first affinity reagent comprising a first hapten-binding partner that is capable of specific binding to the first type of unlabeled nucleotides; and
a second affinity reagent comprising a second hapten-binding partner that is capable of specific binding to the second type of unlabeled nucleotides;
wherein the first affinity reagent comprises one or more first detectable labels that are excitable by a first excitation light source, the second affinity reagent comprises one or more second detectable labels that are excitable by a second excitation light source, and wherein the one or more first detectable labels are spectrally distinguishable from the one or more second detectable labels; and
wherein one of the first excitation light source and the second excitation light source has a wavelength of about 450 nm to about 460 nm, and the other one of the first excitation light source and the second excitation light source has a wavelength of about 520 nm to about 540 nm.

52.-80. (canceled)

81. A labeled nanoparticle, comprising:
a polymer matrix comprising a plurality of detectable labels, wherein the backbone of the polymer comprises one or more cleavable moieties, and wherein the labeled nanoparticle is degradable into smaller polymeric chains upon cleavage of the cleavable moieties.

82.-90. (canceled)
